(19) United States
(12) Patent Application Publication
(10) Pub. No.: US 2019/0161229 A1
(43) Pub. Date: May 30, 2019

(54) APPARATUS AND METHOD FOR IMPROVING SAFE IDENTIFICATION, SELECTION, DISTRIBUTION, AND ADMINISTRATION OF MEDICATIONS AND MEDICAMENTS TO PATIENTS

(71) Applicant: Athenex, Inc., Buffalo, NY (US)

(72) Inventor: Joseph MASE, Barrington, IL (US)

(73) Assignee: Athenex, Inc.

(21) Appl. No.: 16/204,216

(22) Filed: Nov. 29, 2018

Related U.S. Application Data
(60) Provisional application No. 62/592,934, filed on Nov. 30, 2017.

(51) Int. Cl. B65C 3/08; B65C 9/46; A61J 1/14

(52) U.S. Cl. CPC B65C 3/08; B65C 9/46; A61J 1/14; A61J 2205/30; A61J 2205/10; A61J 2205/20; A61M 2205/6081

(57) ABSTRACT

The present application relates to an apparatus and method for improving safe identification, selection, distribution, and administration of medications and medicaments to patients.

### RELATED APPLICATIONS

This application claims priority to U.S. Provisional Application Ser. No. 62/592,934 filed Nov. 30, 2017 entitled “Apparatus and Method for Improving Safe Identification, Selection, Distribution, and Administration of Medications and Medicaments to Patents,” which is hereby incorporated by reference in its entirety.

### FIELD OF THE PRESENT DISCLOSURE

The present application relates to an apparatus and method for improving safe identification, selection, distribution, and administration of medications and medicaments to patients.

### BACKGROUND

Common practice in the medical and veterinary industries is to identify, select, distribute, and/or administer medications and medicaments to patients (e.g., human and animal patients) in order to properly treat medical diseases, disorders, and/or injuries. In a healthcare environment, such as a medical or veterinary hospital, medications and medicaments are readily and routinely stocked, stored, identified, selected, disbursed, distributed, and administered to patients. In all medical environments, particularly in a medical and/or veterinary hospital, operating room, or emergency room, storage, identification, selection, retrieval, disbursement, distribution, and/or administration of the proper type and dose of medication is critically important and directly correlated to positive patient prognosis and patient viability. More specifically, selection, distribution, and administration of medications and medicaments to patients during medical and veterinary visits and admissions require easy, fast, and accurate identification of the proper type and dose of the medication or medicament to be administered to a patient by medical and untrained personnel.

Existing technologies to aid identification of the proper type and dose of medications and medicaments, such as for parenteral administration, comprise labeling the body of a container (e.g., a tube, vial, syringe, IV bag, bottles, oral solution containers or other types of packaging, cartridges, autoinjectors, and eye drops) containing the medication or medicament. For example, a syringe may have a label on the body of the syringe that specifies the name and/or class of a drug medication or compound comprised within the syringe. In addition, the label on the syringe may comprise the color associated with the drug compound or class of drugs comprised therein as designated by the American Society of Testing and Materials International (ASTM).

While the U.S. Food and Drug Administration (FDA) does not require ASTM colors and/or drug classes (e.g., admixture drug classes) on drug product labeling, the ASTM colors and drug classes are often used in medical and veterinary settings. In particular, medical personnel often incorporate labels having ASTM information (e.g., color and drug class) on syringes or IV bags comprising specific drug medications when those compounds are to be admixed to create particular types or concentrations of drug mixtures for administration to patients.

Present syringes comprising specific drug medications or compounds of a particular drug class are typically labeled with the same ASTM color associated with that specific drug class. Thus, syringes comprising different types and doses of drug medications or compounds in the same drug class may be labeled very similarly (e.g., with the same ASTM color code indicating the drug class). In the busy and rapid pace of an emergency room environment or other medical environments, the wrong type or dose of a drug medication or medicament can easily be identified, selected, distributed, and/or administered to a patient by the simple mistake of human error. As one example, certain non-depolarizer drugs such as Atracurium, Cisatracurium, Mivacurium, Pancuronium, Rocuronium, and Vecuronium all look alike, having a fluorescent red color, and sound alike when pronounced, such that often the products get confused. Such drugs in the same class also may have the same concentration, and may further use the same ASTM color as the key identifier for each drug making it easy for them to get confused. Such errors often result in adverse effects, serious side effects, and/or death for the patient.

Further, in the medical and/or veterinary fields, medical personnel are often required to expeditiously retrieve an accurate type, dose, or volume of prescription drug medications and medicaments from dispensary compartments. Labels of existing medications or medicaments comprised in a dispensary compartment must be visible by a user in order for the user to properly identify the type or dose of medication to be retrieved for patient administration. Since existing syringes are not typically labeled on their caps, but only on the body of the syringe, it is often difficult for medical personnel to properly identify, select, distribute, and/or administer a medication or medicament from a dispensary compartment unless that medication or medicament lies horizontally, such that the label on the syringe body is exposed and/or visible to a user.

Accordingly, the existing practice of the medical and veterinary industries to rely on vials, tubes, syringes, and IV bags comprising a drug medication or medicament and a label indicating an ASTM color code and corresponding information that is only located, positioned, or placed on the body of the tube or syringe leads to unnecessary errors in identification, differentiation, selection, distribution, and administration of the corresponding drug medications and medicaments. This type of error is mitigated, reduced, and/or prevented by the apparatus and method of the present disclosure.

### SUMMARY OF THE INVENTION

The present disclosure is directed to an apparatus and method for improving safe identification, selection, distribution, and administration of medications and medicaments to patients. An apparatus of the present disclosure relates to improving administration of a medication or medicament to patients. The apparatus comprises a container (e.g., a vial, syringe, cartridge, IV bag, multichamber bag, pill bottle, oral solution container, and autoinjector), a cap, one or more labels, wherein the cap of the apparatus comprises a label. The vial of the apparatus may also comprise a label. One aspect of the invention is a cap label. A separate aspect of the invention is providing a label with an ASTM color used as a base color of the label to identify the drug class, and further having a unique label design for each drug, with a unique color combination for each strength of drug within each drug family. The invention may at least comprise either aspects independently, or these two aspects in combination.

The label of the present apparatus may be affixed to the outer portion of the cap or the outer portion of the container of the apparatus. In another embodiment, the label may be affixed to the outer portion of the cap and the outer portion of the container of the apparatus. In yet another embodiment, the container of the apparatus further comprises the medication or the medicament. In a further embodiment, labels and/or a color or pattern useful for identification may also be attached to a plunger rod of a syringe. In a further embodiment, a label, color, or pattern useful for identification may be used or added to the rubber portion of a syringe plunger. The label, color, and/or pattern of the plunger rod and/or the syringe plunger may be selected as the same, similar, or complementary to further assist in medicament, class, content, concentration, and/or type identification, without limitation. In a further embodiment, printing technologies may be utilized such as direct printing onto the packaging or by affixing a preprinted label that may be shrink-wrapped onto a package, or by tying or clipping on a label that might act as a flag.

The label of the present apparatus may comprise a color code. The color code on the label may correspond to the ASTM drug class to which the medication or medicament belongs. In addition, the color code on the label may correspond to the drug volume or the drug dose of the medication or medicament comprised therein. Further, the label may comprise information selected from the group consisting of one or more color codes, the total drug content, the drug volume, the final drug dose, the National Drug Code (NDC) (e.g., NDC 70860-XXX-00), the volume of the container, the pharmacy from which the drug was obtained, storage conditions, lot number, expiration date of the drug, and the name and/or location of the container manufacturer.

The label of the present apparatus may also comprise one or more sublabels. The one or more sublabels may comprise a first sublabel. The one or more sublabels may also comprise a second sublabel. Further, the one or more sublabels may comprise a first sublabel and a second sublabel.

The first sublabel and the second sublabel may also comprise information selected from the group consisting of one or more color codes, the total drug content, the drug volume, the final drug dose, the National Drug Code (NDC; e.g., NDC 70860-XXX-00), the volume of the container, the pharmacy from which the drug was obtained, storage conditions, lot number, expiration date of the drug, and the name and/or location of the container manufacturer.

The apparatus of the present disclosure improves the safe identification, selection, distribution, and administration of medications and medicaments to patients. In addition, the cap of the apparatus further comprises a tamper evident cap, a tamper evident seal, or a tamper evident label.

In one embodiment of the present apparatus, the container may further comprise a plunger rod. The plunger rod of the present apparatus may also comprise a label. The label of the plunger rod of this embodiment may be internally embedded within the plunger rod. The label comprised by the plunger rod of this embodiment may further comprise information selected from the group consisting of one or more color codes, the total drug content, the drug volume, the final drug dose, the National Drug Code, the volume of the container, the pharmacy from which the drug was obtained, storage conditions, lot number, expiration date of the drug, and the name and/or location of the container manufacturer. More specifically, the label of the plunger rod of the apparatus may comprise a color code, such as the ASTM color code for drug admixture.

The present disclosure also relates to a method of producing the present apparatus, wherein the method may comprise imprinting colors, designs, or lettering on the label. The present method may further comprise attaching the label to the cap or the container of the apparatus. Further methods are contemplated in the invention utilizing printing technologies to print a label, color, and/or pattern directly onto packaging materials.

The label of the present method may be attached to the outer portion of the cap or the outer portion of the container of the apparatus. The label of the present method may also be attached to the outer portion of the cap and the outer portion of the container of the apparatus. In the present method, the inner portion of the container may further comprise the medication or medicament.

The present disclosure further relates to a method of using the present apparatus by a user. The present method of using the apparatus may comprise visualizing the colors, designs, or lettering on the label. In addition, the colors, designs or lettering on the label of the present methods improve identification, selection, distribution, and administration of medications and medicaments to patients.

A method of dispensing the apparatus of the present disclosure by a scanning device is also described herein. The method of dispensing the apparatus comprises vertically positioning the apparatus in a dispensary compartment. The method further comprises reading the colors, codes, designs, or lettering on the label of the cap, wherein the colors, codes, designs, or lettering on the label of the cap improve identification, selection, distribution, and administration of medications and medicaments to patients. Further, the method of dispensing comprises retrieving the apparatus from the dispensary compartment, and dispensing the apparatus for administration to a patient.

Finally, the present disclosure relates to a method of dispensing the apparatus by a user. The method comprises dispensing the apparatus by vertically positioning the apparatus in a dispensary compartment. The method further comprises reading the colors, codes, designs, or lettering on the label of the cap, wherein the colors, codes, designs, or lettering on the label of the cap improve identification, selection, distribution, and administration of medications and medicaments to patients. Further, the method of dispensing comprises retrieving the apparatus from the dispensary compartment, and dispensing the apparatus for administration to a patient. In further embodiments, the method of dispensing may be utilized by healthcare professionals to further reduce the risk of error in dispensing a wrong type of, or class of, medicament. For example without limitation, a healthcare professional will be able to determine the type or class of medicament through the labeling identification on the packaging and/or cap of a container, and thereby know based on the context of the patient's medical condition or needs at that time whether the selected medicament is appropriate. In one embodiment, a healthcare professional knowing that a patient would be waking up soon or regaining consciousness would be able to quickly identify that the container did not have a label associated with a class of sedatives (or, if a medicament was chosen in error and happened to be a sedative then a healthcare professional would know that an error was made because the label is another visual cue that the wrong medication was selected). Such an embodiment of the invention may help during surgical procedures where the anesthesiologist or healthcare professional providing such services has numerous drugs being used during a procedure that are sitting side by side.

Accordingly, the present disclosure is related to an apparatus and methods that results in an improvement of overall safety during healthcare treatment and administration of medications or medicaments to human and veterinary patients.

### BRIEF DESCRIPTION OF THE DRAWINGS

A brief description of the drawings is as follows.

FIG. 1 is a table showing the American Society of Testing and Materials International (ASTM) Drug Classes, Examples of Drugs, Pantone Color, and Label Examples.

FIG. 2A is a diagram showing a standard, unlabeled container, i.e., syringe and a white, unlabeled cap.

FIG. 2B are figures showing four existing syringes labeled with the ASTM color code indicating the ASTM drug class, the drug name, and/or the drug dose associated with medications Neostigmine (i.e., red and white diagonal stripe), Glycopyrrolate i.e., green), Rocuronium (i.e., red), and Fentanyl (i.e., light blue). Medications or medicaments in the same admixture drug class would have similar or the exact same colors making them difficult to differentiate (e.g., Rocuronium, Atracurium, Cisatracurium, Mivacurium, Pancuronium, and Vecuronium would all be labeled the same). Moreover, such drugs may sound the same when pronounced (e.g., because they end in “IUM” or “CURIUM” or “RONIUM”), and may have the exact same concentration, i.e., they are dosed in similar strengths. In addition, the caps on existing syringes are not labeled with the ASTM color code indicating the ASTM drug class, the drug name, and/or the drug dose at all (as shown in FIG. 2A). Alternatively, the white caps on existing syringes are labeled with the manufacturer's brand and shown /or logo as here.

FIG. 3A are figures showing an embodiment of a cap and a corresponding syringe of the present disclosure both labeled with the ASTM color (i.e., green) and the drug name (i.e., Glycopyrrolate). In addition, the syringe is labeled with the total drug content (0.6 mg), the drug volume (3 ml), and the final drug dose. In this particular embodiment, each concentration of dosage has its own packaging color to identify each different strength within the same drug family.

FIG. 3B are figures showing an embodiment of a cap and a corresponding syringe of the present disclosure both labeled with the ASTM color and the drug name (i.e., Neostigmine). In addition, the syringe is labeled with the total drug content , the drug volume, and the final drug dose. In this particular embodiment, each concentration of dosage has its own packaging color to identify each different strength within the same drug family.

FIG. 3C are figures showing five embodiments of caps of the present disclosure, with corresponding syringes (not shown), labeled with different designs comprising the ASTM color and the drug name for Succinylcholine (i.e., red and black colors and letter), Neostigmine (red and white diagonal stripes with black or white lettering), and Glycopyrrolate (green and white cap with black lettering). Text and/or lettering on the label of the present apparatus, such as on the cap, may be in any size, font, orientation, boldness, etc. in order to aid differentiation between apparatuses comprising different drugs medications and/or medicaments within the same drug classes.

FIG. 4A are figures showing three embodiments of labels for caps of the present disclosure, with corresponding syringes (not shown), that are labeled with different designs comprising the ASTM color (green), along with horizontal or diagonal black and white lettering of the drug name, Glycopyrrolate.

FIG. 4B are figures showing two embodiments of labels for caps of the present disclosure, with corresponding syringes (not shown), labeled with different designs comprising the ASTM color (red), along with vertical or horizontal black and red lettering of a drug name. Typically, each drug will have the same design for the cap. Those drugs in the same drug class may have different labeling designs using the same ASTM base color. In this particular embodiment, the lettering indicates Succinylcholine. Cap labels may be used for any drug containers that require caps such as without limitation oral drugs, dermatological drugs ointments, creams, eye drops, and ear drops.

FIG. 4C is a figure showing three embodiments of labels for caps of the present disclosure, with corresponding syringes (not shown), labeled with different designs comprising the ASTM color (red and white diagonal stripes), along with vertical or horizontal black and red lettering of the drug name, Neostigmine. Typically, each drug will have the same design for the cap. Those drugs in the same drug class may have different labeling designs using the same ASTM base color.

FIG. 5A is a schematic of an embodiment of a label for a syringe, with corresponding cap (see FIGS. 5B and 5C), labeled with the ASTM color (red and white diagonal stripes), along with vertical or horizontal black and red lettering of the drug name, Neostigmine. Here, the syringe is labeled with the total drug content (5 mg), the drug volume (5 ml), and the final drug dose (i.e., 1 mg per ml). In addition, the specific concentration and/or volume of Neostigmine (i.e., 5 mg per 5 ml) is indicated with a first sublabel around the drug name that is colored blue and located at the top of the syringe label.

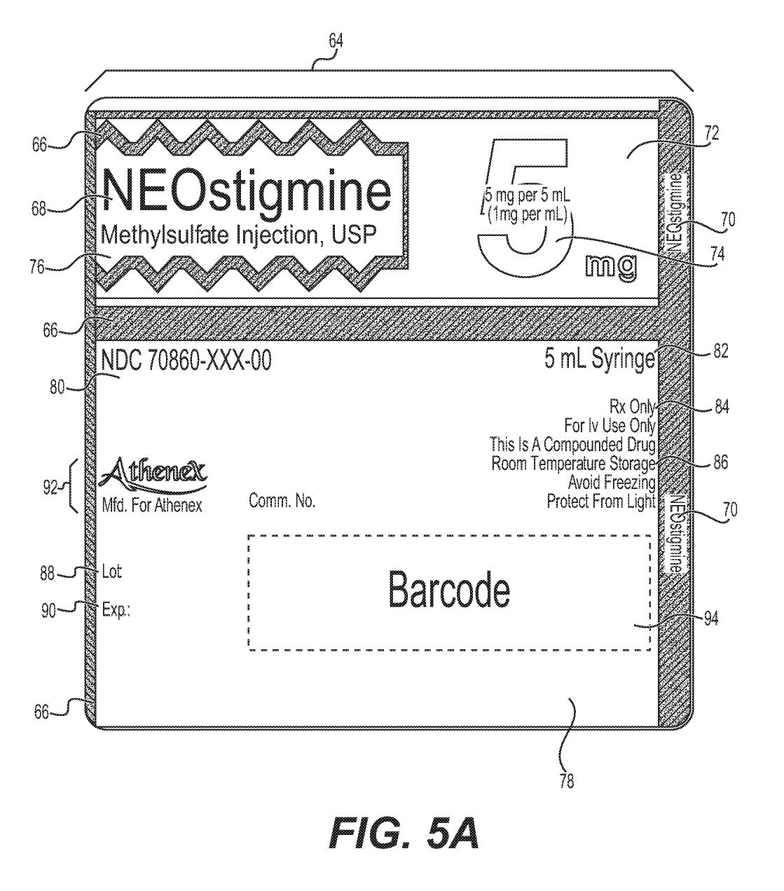

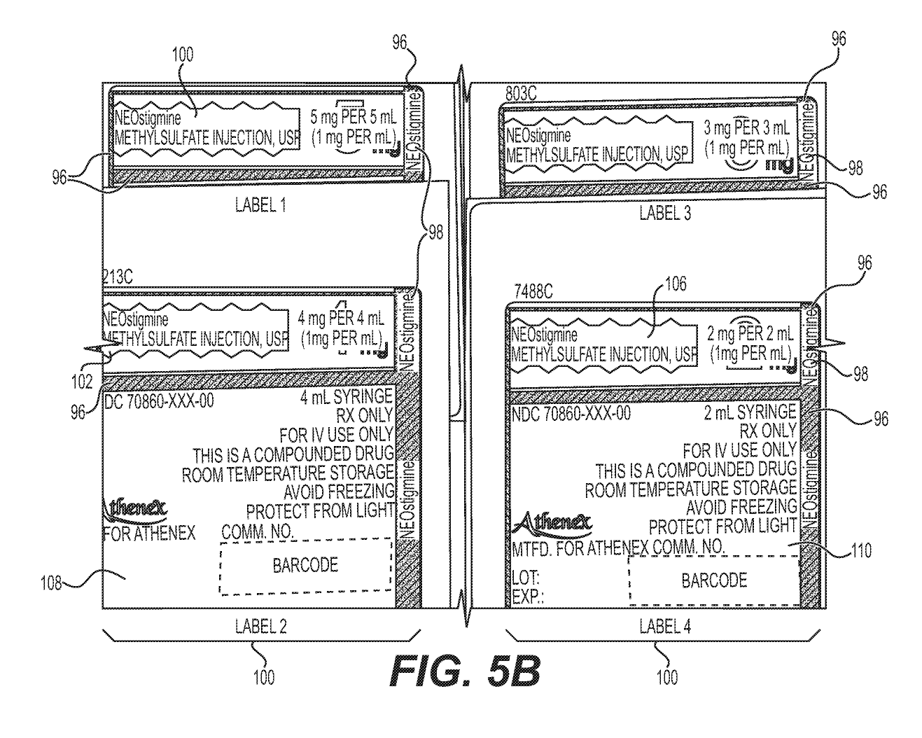

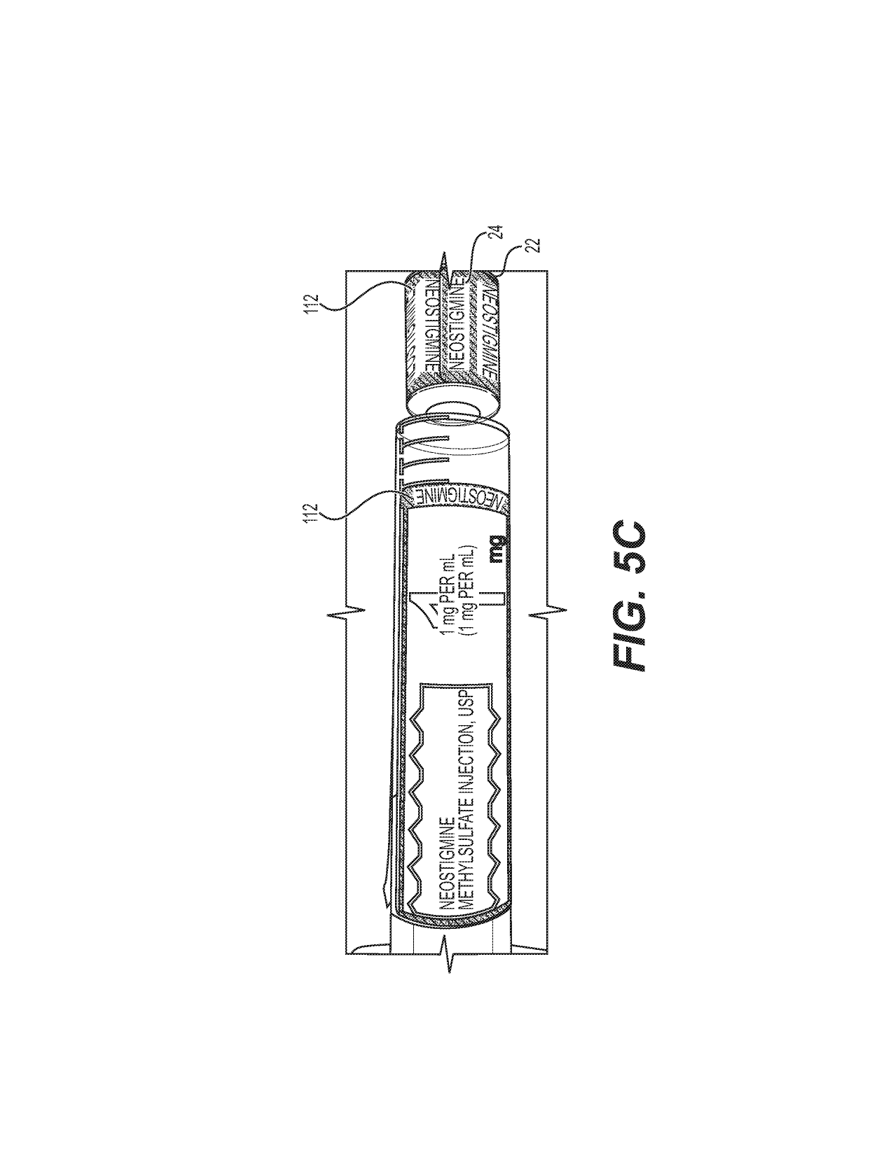

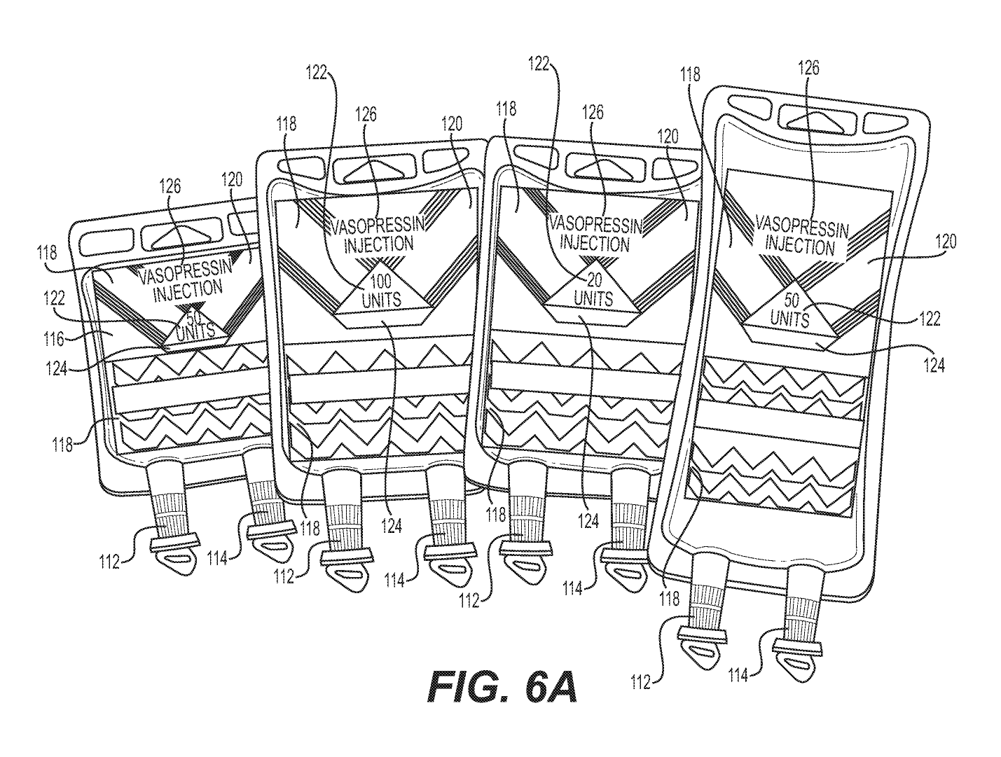

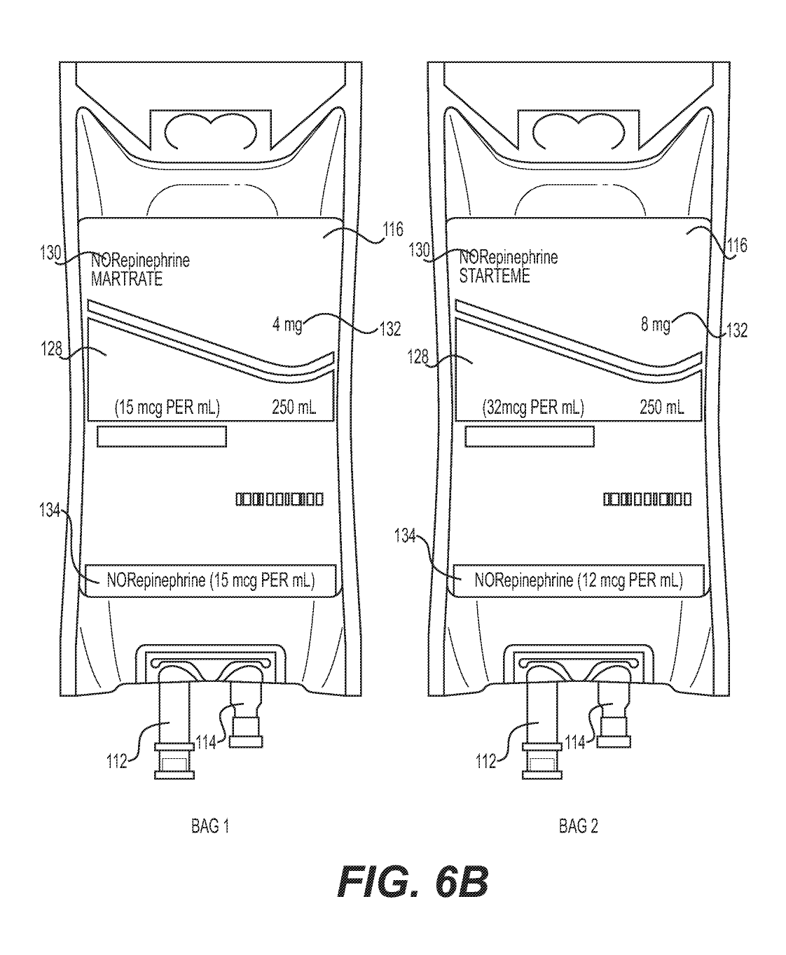

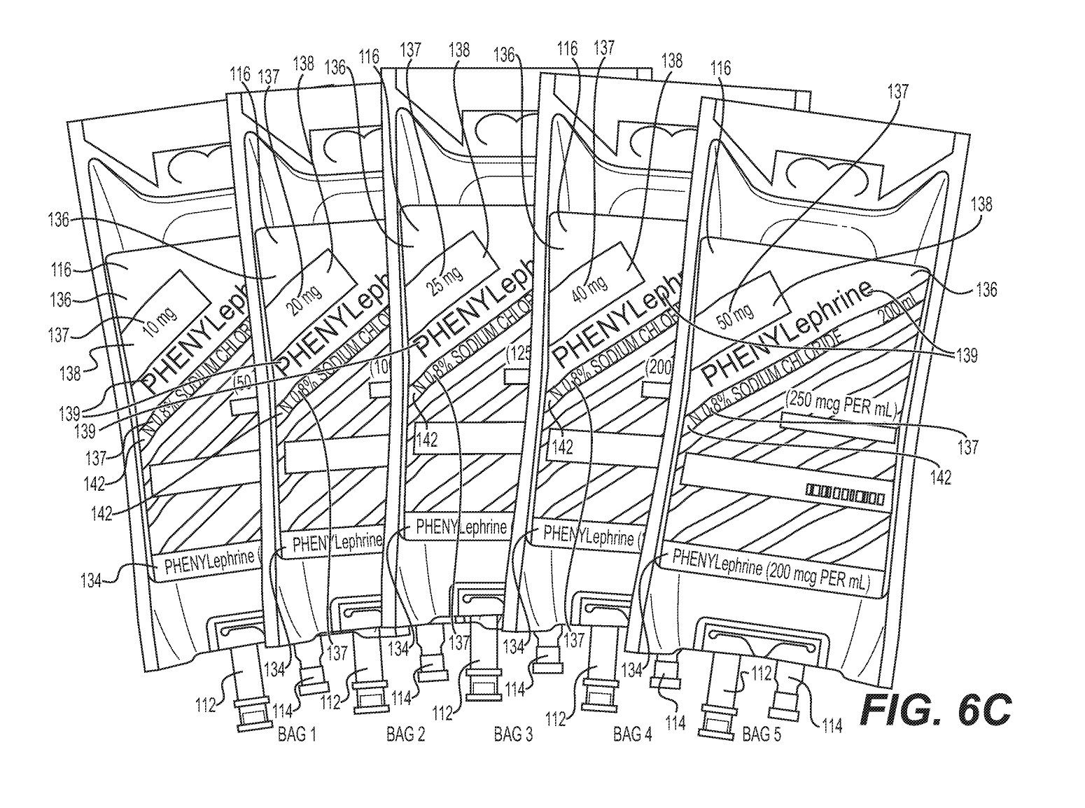

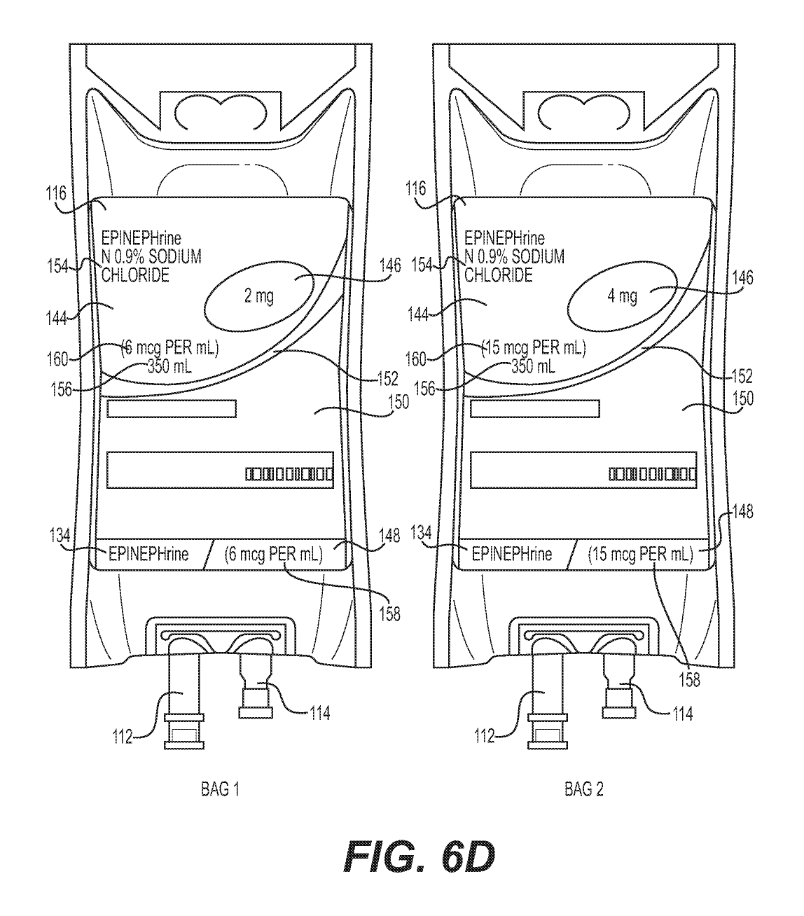

As shown in FIG. 5A, a second sublabel located below the first sublabel of this syringe label embodiment further comprises the following information related to the drug medication or medicament comprised therein: 1) National Drug Code (NDC; e.g., NDC 70860-XXX-00), 2) the volume of the syringe (e.g., 5 ml), 3) the pharmacy from which the drug was obtained (e.g., Rx), 4) storage conditions (e.g., room temperature storage, avoid freezing, protect from light, etc.), 5) lot number, 6) expiration date of the drug, as well as 7) the syringe manufacturer (e.g., Athenex). Typically, the ASTM color would be contained in at least some configuration throughout the entire label to depict a class of drugs. Finally, the label embodiment comprises space for an optional barcode that comprises, consists essentially of, or consists of any scannable or electronically readable information about the medication or medicament that is deemed necessary by a user or manufacturer of the present apparatus.

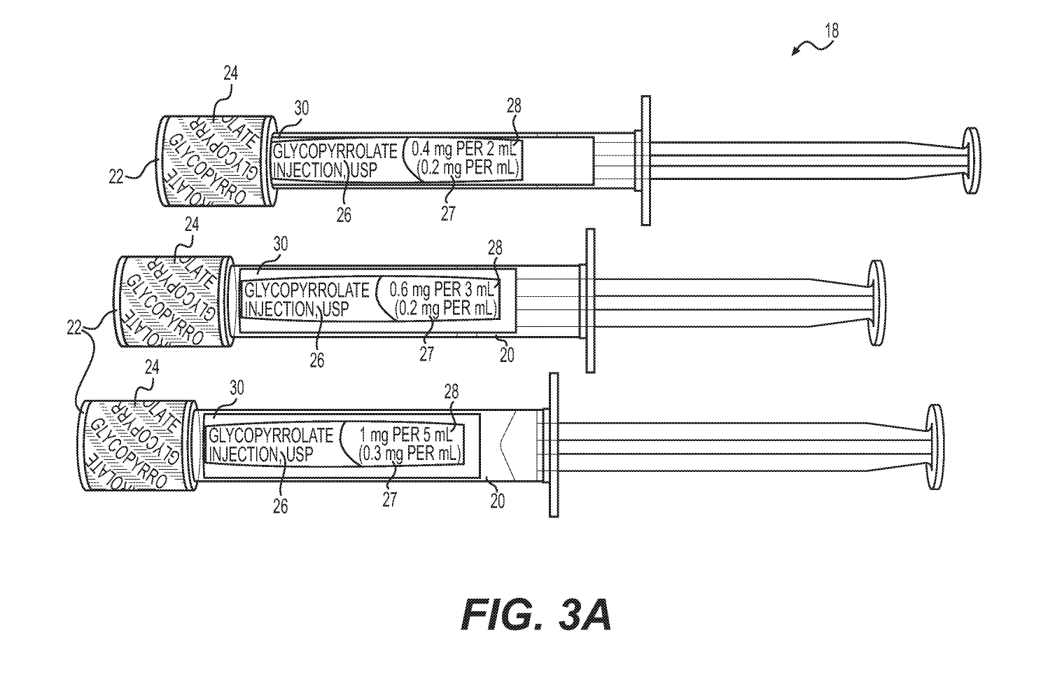

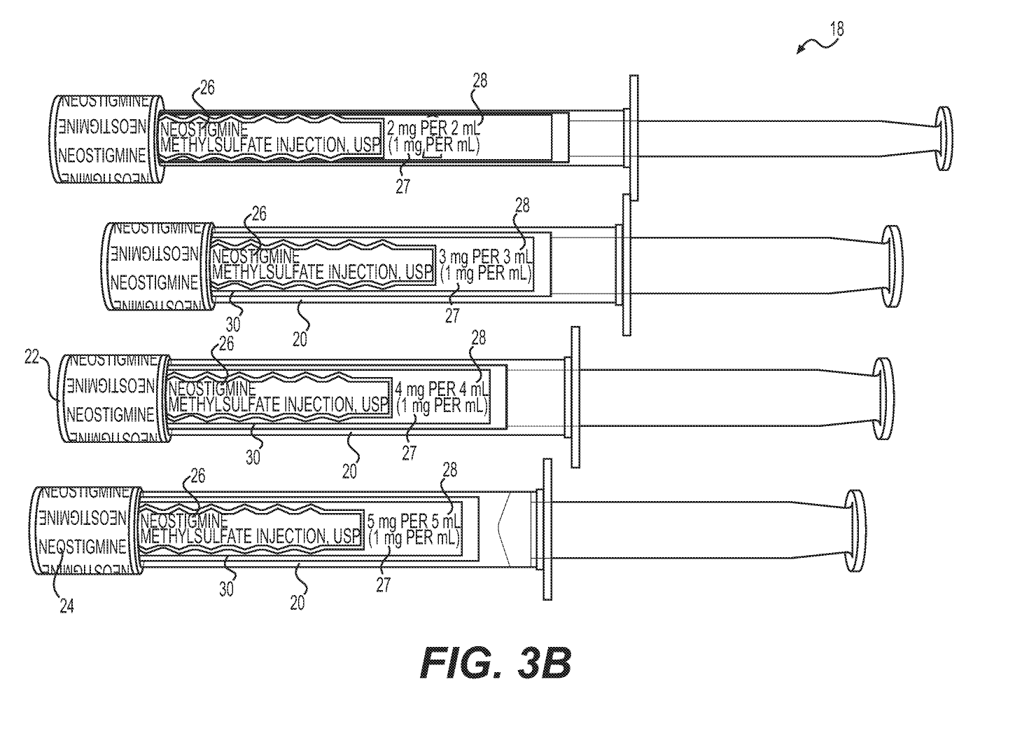

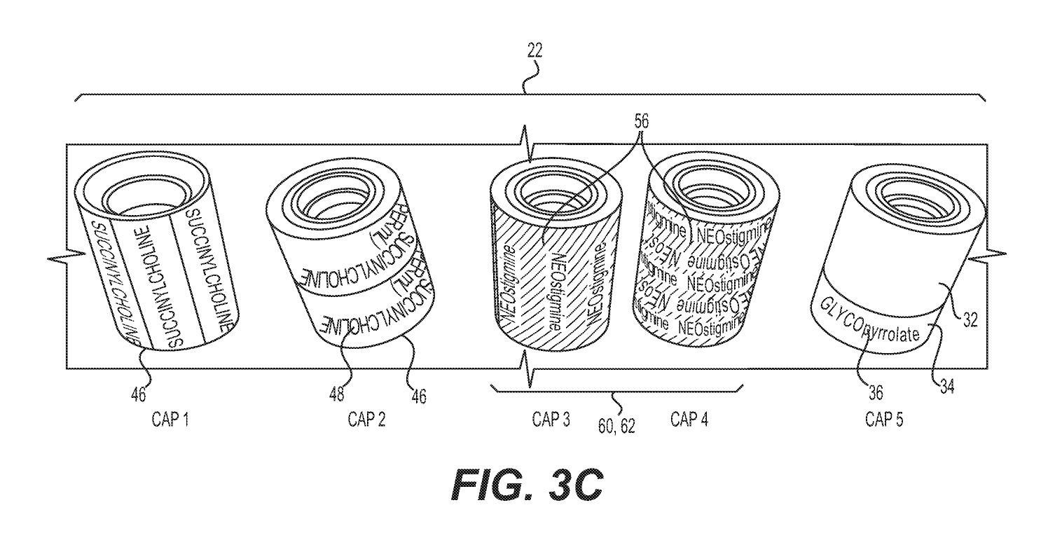

FIG. 5B are figures of four embodiments of a label for a syringe, with corresponding cap (see FIGS. 3A and 5C), comprising the ASTM color (red and white diagonal stripes), along with vertical or horizontal black and red lettering of the drug name, Neostigmine. In addition, these embodiment comprise all of the information specified in FIG. 5A above.

Here, one embodiment of the label of the present disclosure, a first sublabel is colored blue around the drug name, and comprises the total drug content (5 mg), the drug volume (5 ml), and the final drug dose (i.e., 1 mg per ml) as shown in FIG. 5A. In another embodiment, the first sublabel is colored red around the drug name, and comprises the total drug content (4 mg), the drug volume (4 ml), and the final drug dose (i.e., 1 mg per ml). An additional embodiment of the first sublabel is colored yellow around the drug name, and comprises the total drug content (3 mg), the drug volume (3 ml), and the final drug dose (i.e., 1 mg per ml). A final sublabel embodiment exemplified herein comprises the first sublabel that is colored green around the drug name, and comprises the total drug content (2 mg), the drug volume (2 ml), and the final drug dose (i.e., 1 mg per ml). By not using the typical ASTM color as the precise color for each strength and drug in that class, one aspect of the invention permits the drug to have a unique looking and color code for every single product and strength within that drug classes to reduce medication errors and improve patient safety. Having a dosing mistake can be as critical as giving a wrong drug in some cases, i.e., a patient may get an over dose or an under dosage if not given correctly.

Each of the syringe label embodiments of FIG. 5B comprise a second sublabel located below the first sublabel. The second sublabel further comprises the following information related to the drug medication or medicament comprised therein: 1) National Drug Code (NDC; e.g., NDC 70860-XXX-00), 2) the volume of the syringe (e.g., 5 ml), 3) the pharmacy from which the drug was obtained (e.g., Rₓ), 4) storage conditions (e.g., room temperature storage, avoid freezing, protect from light, etc.), 5) lot number, 6) expiration date of the drug, as well as 7) the syringe manufacturer (e.g., Athenex). Finally, the label embodiment comprises space for an optional barcode that comprises, consists essentially of, or consists of any scannable or electronically readable information about the medication or medicament that is deemed necessary by a user or manufacturer of the present apparatus.

FIG. 5C is a figure showing an embodiment of the apparatus of the present disclosure comprising a cap and a corresponding both labeled with the ASTM color (i.e., red and white diagonal strips) and the drug name (i.e., Neostigmine). This embodiment of the present apparatus comprises a label on the cap that further comprises alternating, horizontal lettering in white and black.

The label embodiment of the present apparatus shown in FIG. 5C comprises a first sublabel and/or a second sublabel (not shown). The first sublabel of the illustrative embodiment is colored orange around the drug name, Neostigmine. In addition the first sublabel indicates the total drug content (1 mg), the drug volume (1 ml), and the final drug dose (i.e., 1 mg per ml) comprised by apparatus (see FIG. 5C). The second sublabel (not shown) may further comprise, consist essentially of, or consist of the following information related to the drug medication or medicament comprised therein: 1) National Drug Code (NDC; e.g., NDC 70860-XXX-00), 2) the volume of the syringe, 3) the pharmacy from which the drug was obtained, 4) storage conditions, 5) lot number, 6) expiration date of the drug, as well as 7) the syringe manufacturer. Finally, the label embodiment comprises space for an optional barcode that comprises, consists essentially of, or consists of any scannable or electronically readable information about the medication or medicament that is deemed necessary by a user or manufacturer of the present apparatus. Further, having the cap ASTM colors on both the cap and the label helps to identify the drug class when the cap is removed.

FIGS. 6A-6D are embodiments comprising groups of intravenous bags of the same active ingredient but comprising different coloring configurations based on their different doses.

### DETAILED DESCRIPTION

The present disclosure relates to an apparatus and method for improving safe identification, selection, distribution, and administration of medications and medicaments to patients. In addition, the apparatus of the present disclosure aids the ease, ability, and safety of identification, selection, distribution, and administration of medications and medicaments by users, such as medical personnel in treating patients, such as human and/or veterinary patients. The apparatus of the present disclosure is further capable and intended to hold and/or store medications and medicaments for subsequent identification, selection, distribution, and administration of the medications and medicaments to patients.

The “medication,” “drug medication,” and/or “medicament” described in the present disclosure may be any composition comprising an amount of a pharmaceutically active ingredient and/or drug compound that is known or thought to be efficacious against any medical disease, disorder, and/or injury. The medication and/or medicament may be formulated in various ways well known to one of skill in the pharmaceutical arts for administration to a patient, such as a human or a veterinary patient. The medication or medicament may be administered to a patient in any form, including but not limited to a solid (e.g., a powder), a gel, an ointment, a liquid, an aerosol, or a gas. For example, the drug medication or medicament of the present disclosure may be in solid form, such as in the form of tablets, pills, capsules (including soft gel capsules), cachets, lozenges, ovules, powder, granules, suppositories, and additionally may be in liquid form such as in bottles or containers of oral solutions of different types of medication or medicament. In a typical embodiment, the container has a liquid utilized for injectable products related to healthcare. In other typical embodiments the container has medicaments or other products that may be taken orally. In further typically embodiments, the container has solutions of medicaments or other products related to healthcare.

In various illustrative embodiments of the present disclosure, the medication or medicament (e.g., narcotics) may be administered in liquid form, such as in the form of elixirs, solutions, emulsions, or suspensions. More specifically, the liquid medication or medicament may be administered to a patient parenterally, orally, ophthalmically, nasally, and/or auricularly. For example, the medication or medicament may be administered to a patient via release of an ophthalmic solution into the eye and/or via an auricular solution into the ear, such as by a dropper. In addition, the medication or medicament may be administered via inhalation into the nose. In addition, aqueous suspensions, solutions, elixirs, and emulsions suitable for oral administration of the medication or medicament may be combined with various sweetening or flavoring agents, coloring matter or dyes, with emulsifying and/or suspending agents and with diluents such as water, ethanol, propylene glycol, and glycerin, and combinations thereof.

In exemplary embodiments of the present disclosure, parenteral formulations of the medication or medicament may be suitably formulated as a sterile non-aqueous solution or as a dried form to be used in conjunction with a suitable vehicle or carrier. Vehicles or carriers of the medications and medicaments of the present disclosure are materials or compositions involved in carrying or transporting an active ingredient, compound, analog, or derivative from one location to another location. Carriers may be combined with one or more active compounds to form a treatment.

Treatment carriers of the present disclosure may comprise liquids, gases, oils, solutions, solvents, solids, diluents, encapsulating materials, or chemicals. For example, a liquid carrier of the present disclosure may comprise water (e.g., sterile, pyrogen-free water), buffer, saline solution, a solvent, etc. Gas carriers of the present apparatus may comprise nitrogen, oxygen, carbon dioxide, sulfur dioxide, and other gases.

Preparation of parenteral formulations of the present medications and medicaments under sterile conditions, for example, by lyophilization, may readily be accomplished using standard pharmaceutical techniques well known to those skilled in the art. An illustrative embodiment of the medication or medicament of the present disclosure is suitable for parenteral administration and is best used in the form of a sterile aqueous solution, which may contain other substances, such as salts or glucose.

Suitable routes for parenteral administration of the medication and medicament of the present disclosure include, but are not limited to, intravascular, intravenous, intraarterial, intramuscular, intranasal (e.g., inhalation), cutaneous, subcutaneous, percutaneous, intradermal, and intraepidermal delivery. Suitable means for parenteral administration of the medication or medicament include, but are not limited to, needles and microneedles, syringes, injectors, infusion techniques, or other devices.

### Apparatus of the Present Disclosure

The apparatus of the present disclosure comprises, consists essentially of, or consists of: 1) a container, e.g., without limitation, a tube or a vial, 2) a cap, and 3) one or more labels (see FIG. 3A). The apparatus of the present disclosure may comprise a first part that is a container which may be used for any and all drug packaging, e.g., without limitation, a vial, a flask, a bottle, syringe, autoinjection container, a tube, a cartridge, and/or a bag, such as an intravenous (IV) bag (collectively and individually referred to as “a vial” or “the vial” to generally mean a container that in certain embodiments may be a vial but without limitation to other types of containers embodied in the invention). The vial of the present disclosure may be any object that is capable of carrying, storing, sealing, and/or dispensing a medication or medicament.

An illustrative embodiment of the first part of the apparatus is a vial, a flask, a bottle, a tube, syringe, a cartridge, and/or an IV bag that may be comprised of glass, plastic (e.g. polymers and copolymers), polypropylene, polystyrene, or any combination thereof (see FIGS. 3A and 5C). The vial, flask, cartridge, tube, syringe, IV bag, or bottle of the present disclosure may be a standard vial, flask, cartridge, tube, IV bag, or bottle used for any purpose (“a vial”). An illustrative embodiment of the vial described herein comprises plastic, and forms a plastic vial. A further embodiment of the vial of the present disclosure comprises glass, such as a glass bottle or a glass vial. Any material used to produce the vial of the present disclosure may also be transparent, clear, and/or tinted, such as tinted with a color (e.g., an amber tint). Plastics typically used may be Polyvinyl chloride (PVC), Polypropylene (PP), Polyethylene-Terephthalate (PET), Polyethylene (High Density) HDPE), Polyethylene (Low Density) LDPE (LLDPE), COC (cyclic olefin copolymer, copolymer plastics, multi-polymer plastics, mono polymer plastics, cyclic olefin polymer, Daikyo Crystal Zenith®, or any combination thereof.

The size, shape, and/or dimensions of the vial, as well as each of the inner and outer portions of the vial, the bottle, the flask, the bag, the cartridge, and/or the tube are unlimited. For example, the vial may be cylindrical, square, round, tubular, and/or any size and shape that may provide convenient storage of the medication or medicament comprised therein.

The vial of the present disclosure may also be reusable, recyclable, or non-reusable (i.e., made for a single use only). While not required, often the vial, flask, cartridge, bottle, IV bag, and/or tube of the present disclosure may be sterilized and/or decontaminated by any standard methods known in the art to reduce, inhibit, or prevent decontamination, degradation, and/or decomposition of the medication or medicament comprised therein, such as from debris, mold, bacteria, fungus, microorganisms, as well as accidental and/or intentional cross-contamination.

The vial of the present disclosure comprises an outer portion and an inner portion. The inner portion or inner compartment of the vial is capable of carrying, storing, sealing, and/or dispensing a medication or medicament therein. The outer portion of the vial provides a surface upon which further information regarding the medication or medicament may be comprised.

In addition, the vial portion of the apparatus of the present disclosure may comprise, consist essentially of, consist of, or be formed to accompany and/or attach to one or more additional component. Additional components that the vial may comprise, consist essentially of, consist of, or be formed to accompany or to attach to include, but are not limited a needle, a microneedle, a nozzle, a plunger, a plunger rod, a bulb, a cap, a label, a seal, a plug, a port, an outlet, a vent, and/or any combination thereof. A port, an outlet, or a vent of the vial of the present apparatus may be closable and/or sealable by a cap, a seal, and/or a plug or unsealed.

In addition, the vial may comprise tubing and/or piping through which the medication or medicament may flow into and/or out of the inner portion of the vial by any means or source of flow known to be used in the medical arts (air, water, solution, gas, etc.). An illustrative example of this embodiment of the present apparatus would be an intravenous (IV) bag. Further, an IV bag comprising a port, a cap, and tubing, wherein the tubing feeds through the port of the IV cap and allows flow of a medication or medicament comprised therein into and out of the IV bag. In certain embodiments, specialized labeling may be employed to indicate combinations of labeling on the IV bag port (similar to labeling configurations in a syringe cap embodiment).

Further, an exemplary embodiment of a vial of the present disclosure is a standard syringe used in the medical and veterinary arts for administering medications and medicaments that comprises a tube, a nozzle, a plunger, a plunger rod, a plunger cap, and/or a needle for injecting or withdrawing liquid compositions, such as via jet injection, air pushing powder, or liquid through the skin (e.g., subcutaneously)

Any size, shape, and/or dimension of a syringe used in the medical arts are sufficient for the present disclosure. An illustrative syringe of the present disclosure comprises a volume of medication and/or medicament that ranges from about 1 to about 200 mls, from about 1 to about 150 mls, from about 1 to about 100 mls (e.g., 1-100 mls syringes). For example the syringe of the present disclosure may comprise a syringe with about a 0.5 ml fill volume, and further from about 5 mls to about 100 mls, from about 10 mls to about 100 mls, from about 10 mls to about 75 mls, from about 1 ml to about 90 mls, from about 1 ml to about 80 mls, from about 1 ml to about 70 mls, from about 1 ml to about 60 mls, from about 1 ml to about 50 mls, from about 5 mls to about 50 mls, from about 1 ml to about 40 mls, from about 5 mls to about 40 mls, from about 1 ml to about 30 mls, from about 1 ml to about 20 mls, from about 1 ml to about 10 mls, from about 1 ml to about 9 mls, from about 1 ml to about 8 mls, from about 1 ml to about 7 mls, from about 1 ml to about 6 mls, from about 1 ml to about 5 mls, from about 1 ml to about 4 mls, from about 1 ml to about 3 mls, from about 1 ml to about 2 mls, about 10 mls, 9 mls, 8 mls, 7 mls, 6 mls, 5 mls, 4 mls, 3 mls, 2 mls, about 1 ml or less than 1 ml of medication or medicament for treatment of patients. In a typical embodiment, a syringe would have about a 1 ml fill volume to about a 10 ml fill volume, a vial could have a fill volume as low as 0.5 ml, and IV bags would have a fill volume from about 25 ml up to 5 liters.

In addition to the vial component, the apparatus of the present disclosure may also comprise a second part that is a lid, a cap, a plug, and/or a seal. An illustrative embodiment of the second part of the apparatus is a cap that may be comprised of glass, plastic (e.g., polymers and copolymers), polypropylene, polystyrene, rubber, or any combination thereof. The cap of the present disclosure may be any component that is capable of attaching to, closing, and/or sealing the vial of the present disclosure.

An illustrative embodiment of the cap described herein comprises plastic, and forms a plastic cap. The cap of the present disclosure may also be reusable, recyclable, or non-reusable (i.e., made for a single use only). Certain syringe caps may comprise plastic, rubber, a rubber material, or a combination of at least two of those materials. Illustrative embodiments of the cap of the present disclosure comprise any type of caps used to close and/or seal a vial comprising a medication or medicament. For example, a cap of the present disclosure may include but is not limited to a syringe cap, a vial cap, a tube cap, a port cap, a vent cap, an outlet, cap, a plug, a pipette tip or a pipette cap, and/or a dropper cap (e.g., such as used for infant medicines or eye and ear drops, etc.). As noted above, similar configurations can also be applied to an IV bag and associated port.

Optionally, the cap of the present disclosure may be a tamper evident cap. The tamper evident cap of the present disclosure may comprise, consist essentially of, or consist of a tamper evident seal and/or a tamper evident label. The tamper evident seal may be internal to the cap, and may extend between and connect the label of the cap and the label of the syringe.

For example, one embodiment of the tamper evident cap comprises, consists essentially of, or consists of a top cap and a seal. In another embodiment, the tamper evident cap comprises, consists essentially of, or consists of a tamper evident label. In a further embodiment, the tamper evident cap comprises, consists essentially of, or consists of a top cap and/or a seal of a tamper evident cap, a tamper evident label, or any combination thereof.

When the top cap, the seal, and/or the tamper evident label of the tamper evidence cap form a single structure while engaged onto the vial, no evidence of tamper with the vial has occurred. When the top cap, the seal, and/or the tamper evident label of the tamper evidence cap are separated while engaged with the vial, there is evidence that the vial has been tampered with.

Typically, evidence of tamper with the vial is indicative that the medication or medicament comprised within the vial should be discarded for safety reasons and concerns. Alternatively, when engaged onto the vial, the tamper evident cap generally aids to reduce, inhibit, and/or prevent decontamination, degradation, and/or decomposition of the medication or medicament comprised within the vial, such as from debris, mold, bacteria, fungus, microorganisms, as well as accidental and/or intentional cross-contamination.

Once removed from the vial (or container, e.g. a syringe) to which it is attached and/or corresponds, the present tamper evident cap and/or tamper evident label may not be used to close or reseal the same vial or a different vial or tube. This is a safety feature of the present apparatus comprising a tamper evident cap and/or tamper evident label. More explicitly, the inability to reapply a tamper evident cap and/or tamper evident label of a potentially used medication or medicament aids to reduce, inhibit, and/or prevent decontamination, degradation, and/or decomposition of the medication or medicament comprised within the vial, such as from debris, mold, bacteria, fungus, microorganisms, as well as accidental and/or intentional cross-contamination.

The apparatus of the present disclosure is typically and properly used by a user, such as medical personnel, when the first part of the apparatus (i.e., the vial) is filled with a medication or medicament. The medication or medicament is administered to the inner portion of the vial, which may comprise or be attached to an additional component (e.g., a needle). The vial then comes in contact with the second part of the apparatus (i.e., the cap) by screwing, twisting, turning, closing or in some manner or fashion locking the cap onto the vial to form a sealed apparatus.

The cap of the sealed apparatus may comprise, consist essentially of, or consist of a tamper evident seal. Once the vial and the cap have fully engaged, the cap of the present disclosure prevents air or additional and/or extraneous compositions from getting into the sealed apparatus and contamination the medication or medicament described therein (e.g., debris, mold, bacteria, fungus, microorganisms, as well as accidental and/or intentional cross-contamination, etc.).

The label of the present apparatus may be any size, and is typically sized to fit around the circumference of one or more portions of the apparatus (e.g., the vial, the plunger rod, and/or the cap). In addition, the label may be made of any material that allows the label to reflect transcribed or imprinted language upon its surface to a user and to adhere to the vial and/or the cap of the apparatus. In certain embodiments, a material with a label or such imprinted language on its surface could be a plungered rod. In certain other embodiments, a plungered rod may be colored to one or more ASTM colors to further distinguish the products. Other color and labeling configurations as described, however, may remain the same. Having such an overall configuration would provide a check against human or other similar types of error for the drug class based on the plunger rod color. The disclosure contemplates similar portions of container with more than one color, or a reversion to an ASTM color as a check against human error. Certain elastomeric plungers may additionally be colored as well in some embodiments. Further, a plunger (e.g., one comprising rubber) may be colored similar to, corresponding with, the same as, or congrued to the plunger rod to aid in identification.

Illustrative embodiments of the label of the present apparatus may comprise, consist essentially of, or consist of paper, plastic (e.g., acetate, vinyl, polymers, copolymers, polyethylene terephthalate, or combinations thereof), woven and non-woven cloth, latex, metal (e.g., foil), wax, resin, shrink band, or any combination thereof. Further, the label of the present apparatus may comprise, consist essentially or, or consist of any kind of label or specialized label, including but not limited to piggyback labels, smart labels (e.g., comprising chips embedded in them), blockout labels (e.g., label is not transparent), radioactive labels, security labels, antimicrobial labels, fold-out labels, barcode labels, etc. Alternatively, the label of the present disclosure may also be printed directly on the present apparatus without the need for an intermediate substrate or material. In further alternative embodiments, the label of the present disclosure may also be engraved, e.g. without limitation, by electromagnetic spectrum energy or mechanical energy (e.g. without limitation, a laser, water jet, or metal) directly on the present apparatus without the need for an intermediate substrate or material.

The label of the apparatus may further comprise an adhesive component or composition that enables attachment of the label to the cap, the vial or any other portion of the apparatus. In an illustrative embodiment, the adhesive component of the label or sublabels of the present apparatus comprises glue, tape, paste, etc. In alternative label or sublabel embodiments, the adhesive component of the present apparatus may comprise, consist essentially of, or consist of rivets, shrink wrap, stitches (e.g., sewing thread), yarn, twine, plastic ties, static cling, etc.

Typically, the adhesive component is located on the back side of the label, which is the side opposite of the front side of the label. Alternatively, the front side of the label of the apparatus may comprise descriptive information transcribed or imprinted thereon for ease of identification, selection, distribution, and administration of medications and medicaments comprised within the apparatus.

In addition, the front side of the label or sublabel may also comprise an overcoat, a laminate, and/or a coating to protect the integrity of the information transcribed thereon. This overcoat or laminate may comprise, consist essentially of, or consist of any material known to allow and/or enable the ease and quick visibility, visualizing, and/or readability of the information comprised on the label or sublabel (clear, tinted, or colored plastic, glass, or combinations thereof).

For example, the label may specify the name and/or class of a drug medication or compound comprised within the apparatus. The label of the present apparatus may be designed, colored, and lettered in any way at all to help ease the identification, selection, distribution, and administration of medications and medicaments comprised therein to users, such as medically trained and untrained personnel. In particular, the labels of the present apparatus may be transcribed, imprinted, and/or formatted to comprise the designs, coloring, and lettering associated with the drug compound or class of drugs comprised therein as designated by the American Society of Testing and Materials International (ASTM).

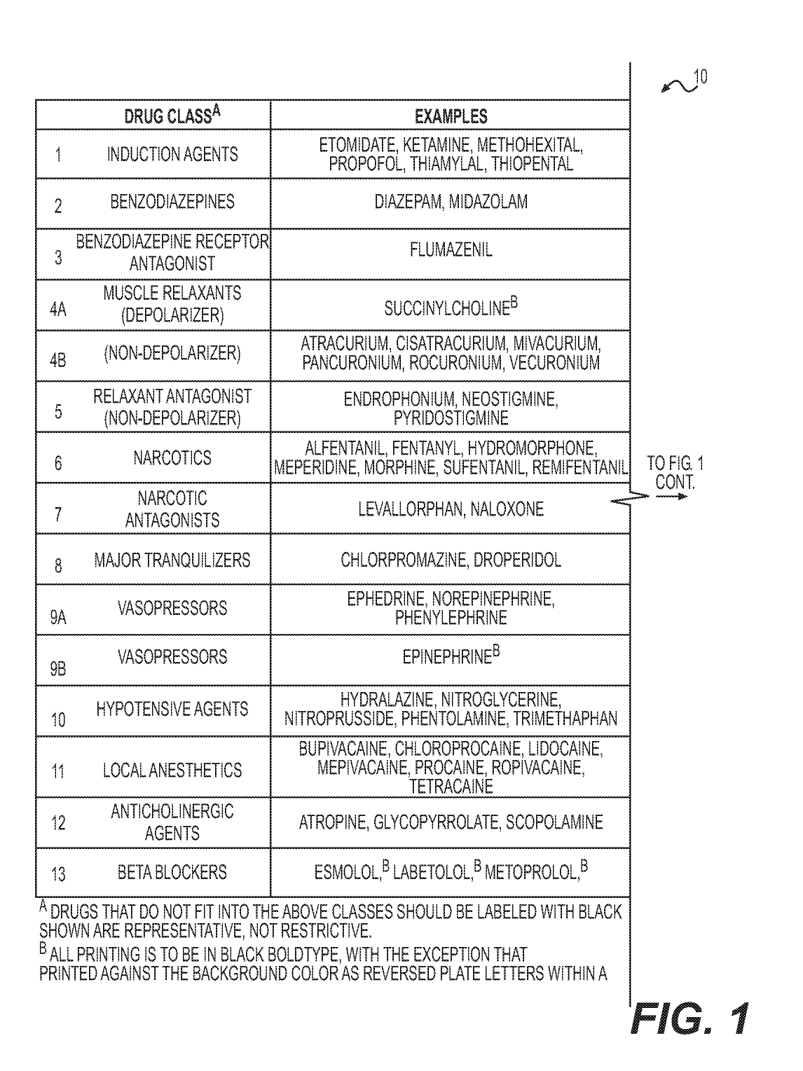

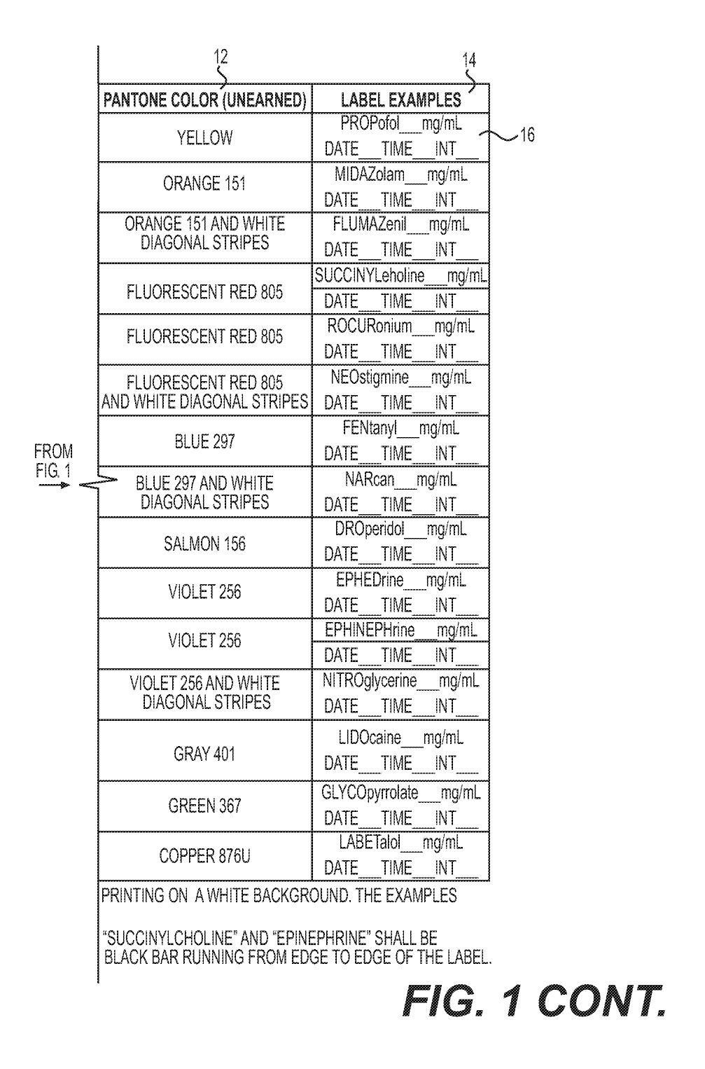

It should be noted that not all drug compounds, classes, and/or medications have been designated an ASTM color code as shown in FIG. 1. As shown in FIG. 1, the ASTM color code is based on the Pantone Matching System (PMS) colors 10. The Pantone system of colors is well known in the printing industry. In FIG. 1, the table column titled “PANTONE COLOR (UNEARNED)” 12 provides a written description of the color code or pattern, and in typical versions of the ASTM color code, a corresponding example of the color and pattern is indicated in the column “LABEL EXAMPLES” 14. Thus, although not depicted in color in the figure herein, a color label configuration example 16 of the written description would be on the ASTM color code chart 10 for medicaments. It is noted that FIG. 1 is a depiction of the ASTM color code, but that the portion stating “ALL PRINTING IS TO BE IN BLACK BOLDTYPE, WITH THE EXCEPTION THAT “SUCCINYLCHOLINE” and “EPINEPHRINE” SHALL BE PRINTED AGAINST THE BACKGROUND COLOR AS REVERSED PLATE LETTERS WITHIN A BLACK BAR RUNNING FROM EDGE TO EDGE OF THE LABEL” is not practiced or embodied in the present invention.

Further, the U.S. Food and Drug Administration (FDA) does not require ASTM colors and/or drug classes on drug product labeling. Thus, drugs that do not fit into the designated ASTM classes may be colored, coded, and or labeled with any color that is not included in the current ASTM designation, such as black printing on a white background (see FIG. 1) or as deemed distinctive by the user. In certain embodiments of the invention, color combinations with or without ASTM colors, or related to but visually distinct from ASTM colors, may be selected and used. Although they are not required or restrictive, the ASTM standards for colors and drug classes are often adopted and used in medical and veterinary settings (see FIG. 1).

In addition to the ATSM information transcribed or imprinted thereon, the label of the apparatus may further comprise information specific to the particular medication or medicament. Text and/or lettering on the label of the present apparatus may be in any size, font, orientation, boldness, etc. in order to aid differentiation between apparatuses comprising different drugs medications and/or medicaments within the same drug classes.

For example, the label of the present apparatus may include additional information not limited to: the National Drug Code (NDC) (e.g., NDC 70860-XXX-00), 2) the volume of the syringe (e.g., 5 ml), 3) the pharmacy from which the drug was obtained (e.g., Rₓ), 4) storage conditions (e.g., room temperature storage, avoid freezing, protect from light, etc.), 5) lot number, 6) expiration date of the drug, as well as 7) the syringe manufacturer (e.g., Athenex). Finally, the label embodiment comprises space for an optional barcode that comprises, consists essentially of, or consists of any scannable or electronically readable information about the medication or medicament that is deemed necessary by a user or manufacturer of the present apparatus. In a further embodiment, the label may comprise an radio frequency identification (RFID) tag that may be scanned in logistics, storage, transit, or during usage in a healthcare setting by a healthcare professional. The invention contemplates other radio detection devices and technology that permit accurate and fast scanning or detection of a label to aid with identification under any circumstance.

The apparatus of the present disclosure further comprises one or more labels, two or more labels, three or more, four or more, five or more, six or more, seven or more, eight or more, nine or more, or a plurality of labels. The label of the present apparatus may comprise, consist essentially of, or consist of one or more sublabels, two or more sublabels, or an unlimited plurality of sublabel. In some embodiments, the total number of labels and sublabels attached or affixed or embedded into the present apparatus may be about 6 or less, about 5 or less, about 4 or less, about 3 or less, about 2 or less, or about 6, about 5, about 4, about 3, about 2, or only about 1 label.

In some embodiments, the number of labels and sublabels attached or affixed to the vial portion of the present apparatus may be about 6 or less, about 5 or less, about 4 or less, about 3 or less, about 2 or less, or about 6, about 5, about 4, about 3, about 2, or only about 1 label. In addition, the number of labels and sublabels attached or affixed to the cap portion of the present apparatus may be about 6 or less, about 5 or less, about 4 or less, about 3 or less, about 2 or less, or about 6, about 5, about 4, about 3, about 2, or only about 1 label. Further, the number of labels and sublabels embedded into any internal portion(s) of the present apparatus may be about 6 or less, about 5 or less, about 4 or less, about 3 or less, about 2 or less, or about 6, about 5, about 4, about 3, about 2, or only about 1 label.

An illustrative embodiment of the present disclosure comprises about two sublabels, including a first sublabel and a second sublabel (see FIGS. 3A and 5C). All information transcribed or imprinted on the label of the present apparatus, may also be transcribed or imprinted on one or more sublabels of the present apparatus. However, the sublabels may also have the same and/or different information transcribed or imprinted thereon in order to comprise the totality of information transcribed or imprinted on the label of the present apparatus for any particular medication or medicament. Excluding limits to the total surface area and/or circumference of the present apparatus to which a label or a sublabel may be affixed or attached, there is no limit to the amount of information that may be transcribed, imprinted, or comprised on the labels or sublabels of the present apparatus.

The apparatus may also comprise one or more labels or sublabels on any portion of the apparatus that may comprise labels or sublabels. More specifically, the cap portion of the apparatus may comprise, consist essentially of, or consist of one or more labels or sublabels. In addition, the vial of the apparatus may comprise, consist essentially of, or consist of one or more labels or sublabels. Notably, in one embodiment of the present apparatus, the outer portion of the vial may comprise one or more labels or sublabels, while the inner portion of the vial may further comprise the medication or medicament for treatment of patients. In a separate embodiment, the outer portion of the container may comprise one or more labels or sublabels, while the inner portion of the vial (e.g., the plunger, the plunger rod, and/or the plunger cap) may also comprise one or more labels or sublabels in addition to the medication or medicament.

An illustrative embodiment of the present apparatus comprises a label on the cap, a label on the vial, or any combination thereof. For example, an exemplary embodiment of the present apparatus comprises a vial and a cap, wherein the cap comprises a label. Another embodiment of the present apparatus comprises a vial and a cap, wherein both the vial and the cap each comprise a label. An additional embodiment of the present disclosure comprises a vial comprising a needle and a cap, wherein both the vial and the cap each comprise a label.

In particular, the cap portion of the apparatus comprises a label indicating the ASTM color (e.g., green) and the drug name (e.g., Glycopyrrolate). In an additional embodiment of the cap of the present disclosure, the label of the cap may further comprise an optional barcode. For example, a cap of the present disclosure may comprise the ASTM color (e.g., green), the drug name (e.g., Glycopyrrolate), a barcode, and/or any combination thereof. The optional barcode comprises, consists essentially of, or consists of any scannable or electronically readable information about the medication or medicament that is deemed necessary by a user or manufacturer of the present apparatus (e.g., the type, dose, concentration, volume, use, drug class, etc. of medication or medicament comprised therein).

The labeled cap of the present disclosure comprising the ASTM color and drug name helps ease the identification, selection, distribution, and administration of medications and medicaments comprised therein for users, such as medical or untrained personnel. Further, when the labeled cap is accompanied, attached to, or affixed to a labeled or unlabeled vial comprising a medication or medicament, the apparatus helps ease the identification, selection, distribution, and administration of medications and medicaments comprised therein.

Further, when the cap is removed, discarded, or separated from the vial, the ASTM color code indicated on the cap continues to identify the accompanied type and dose of medication or medicament comprised within the apparatus. One embodiment of the cap of the present apparatus has the capability of detaching a portion of the cap or the label on the cap that remains behind and/or affixed or attached to the vial after the cap is removed. This embodiment of the cap allows the ASTM color, code, or identification from the cap to remain at the top of the syringe.

Accordingly, one embodiment of the apparatus of the present disclosure comprises a labeled cap with the ASTM color code corresponding to the type and/or dose of drug medication comprised therein, which results in an improvement of overall safety during healthcare treatment administration to human and veterinary patients. These are unexpected benefits of the apparatus of the present disclosure over the existing and/or prior art technologies.

Alternatively, another embodiment of the present apparatus comprises a vial that further comprises a cap, a plunger rod, a plunger cap, and/or a plunger or any combination thereof. A plunger is a physical component located on the inner portion or compartment of the vial. The plunger functionally aids in injecting and/or extracting a medication or medicament from the inner portion of the vial to the external environment, and often into a patient during administration of a medication or medicament. A plunger may comprise one or more plunger rods, and typically comprises a single plunger rod. A single plunger rod is often located in the center of the plunger, so as to connect one, two, or more plunger caps.

Plunger caps are generally located at opposite ends of the plunger rod to form the plunger. For example, one plunger cap of the plunger typically remains external to the vial at one end of the plunger, while the opposite plunger cap remains internal to the vial at the opposite end of the plunger. In illustrative embodiments of the plunger, the one or more plunger caps (e.g., the internal plunger cap) may comprise rubber. Typically, the invention would be embodied in a syringe or other similar container having only one rubber plunger, although as noted above other embodiments are contemplated.

In addition, to the cap of the present apparatus, the plunger rod and/or plunger comprised in the vial of this embodiment of the present apparatus may further comprise, consist essentially of, or consist of one or more labels and/or sublabels. The labels and/or sublabels of the plunger, the plunger rod, and the plunger cap of the vial may be located and/or positioned on the external portion or the internal portion of each of these vial components.

In an illustrative embodiment, the label and/or sublabels of the present disclosure may not be comprised on any additional material at all, such as material affixed or attached to the outer portion of the apparatus components. In such embodiments, the label may be transcribed, imprinted, and/or embedded directly into any internal portion or component of the apparatus. For example, the labels and sublabels of the present apparatus comprising colors (e.g., ASTM colors), codes (e.g., barcodes), shapes, and or lettering corresponding to relevant information about the medication or medicament comprised in the apparatus may be internally transcribed, imprinted, and/or embedded directly into any internal portion or component of the apparatus. Text and/or lettering on the label of the present apparatus may be in any size, font, orientation, boldness, etc. in order to aid differentiation between apparatuses comprising different drugs medications and/or medicaments within the same drug classes.

Illustrative embodiments of portions of the present apparatus comprising internally embedded labels within its components include, but are not limited to a cap, a plunger rod, a port, a plunger, a vial, a bag. An exemplary embodiment of the present apparatus comprises a vial further comprising a plunger, a plunger rod, and/or at least one plunger cap that is internally transcribed or embedded with an internal label comprising the ASTM color code and/or a barcode. Thus, an illustrative embodiment comprises a plunger rod that is fully or partially colored (e.g., by internal transcription or embedding) with an ASTM color.

In certain embodiments, a cap of a certain color (or other removable portion of the broad class of medical device containers disclosed herein) could be removed during ordinary operation by a healthcare provider or assistant. A portion of such cap or removable portion could be designed to remain affixed on the remaining portion of such container. Thus, in this fashion a color or label indicator could remain on the container. This would permit others to understand the configuration of colors used for the container prior to disposal or even after disposal. Furthermore, after use, when such cap or removable portion is removed during ordinary operation, a new color or label could also be revealed. This new color or label could provide extra useful information to a healthcare provider, or even just a quick indication that the container was used already which otherwise could be difficult to quickly see with a high degree of accuracy. In alternative embodiments, a plurality of such features could be combined such that a product with a removable portion would have some of the same portion be retained on a container, and a new color or label would be revealed at the same time. In certain configurations, the colors and/or labels could be configured to cooperate to indicate new information not understood or known by either independently to a medical provider, e.g., about the status or composition of the container.

### Methods of Producing and Using the Present Apparatus

The present disclosure also relates to a method of producing and using the apparatus described herein. More specifically, a method of the present disclosure is related to producing an apparatus according to the description contained herein. In addition, a method of the present disclosure is related to using the apparatus in order to improve safe identification, selection, distribution, and administration of medications and medicaments to patients.

As previously described above, the apparatus of the present disclosure comprises, consists essentially of, or consists of a cap, a vial or a tube, and one or more labels. The one or more labels of the present disclosure may comprise one or more sublabels, including, but not limited to a first sublabel and/or a second sublabel. In addition the labels or sublabels of the present apparatus may be transcribed, imprinted, and/or embedded to comprise any and all information relevant to the medication or medicament or the safety or the medication or medicament comprised within the apparatus.

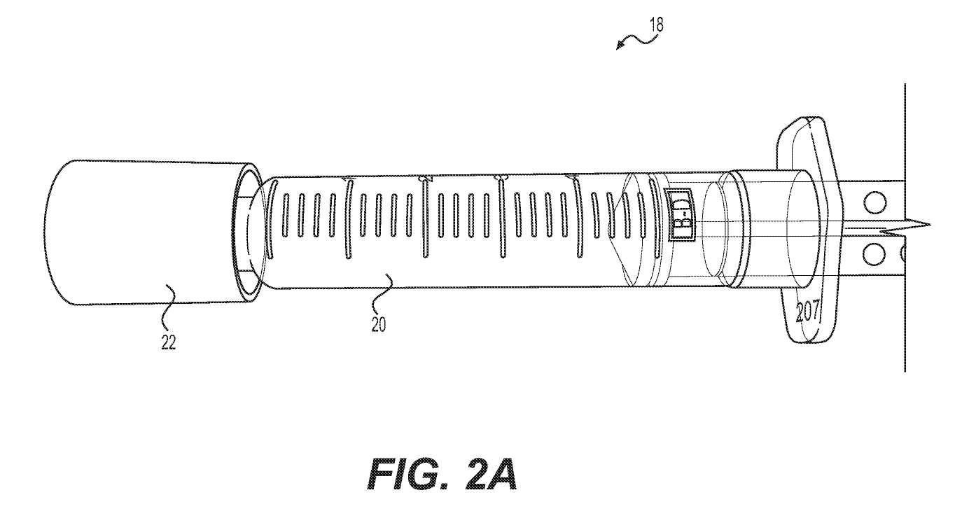

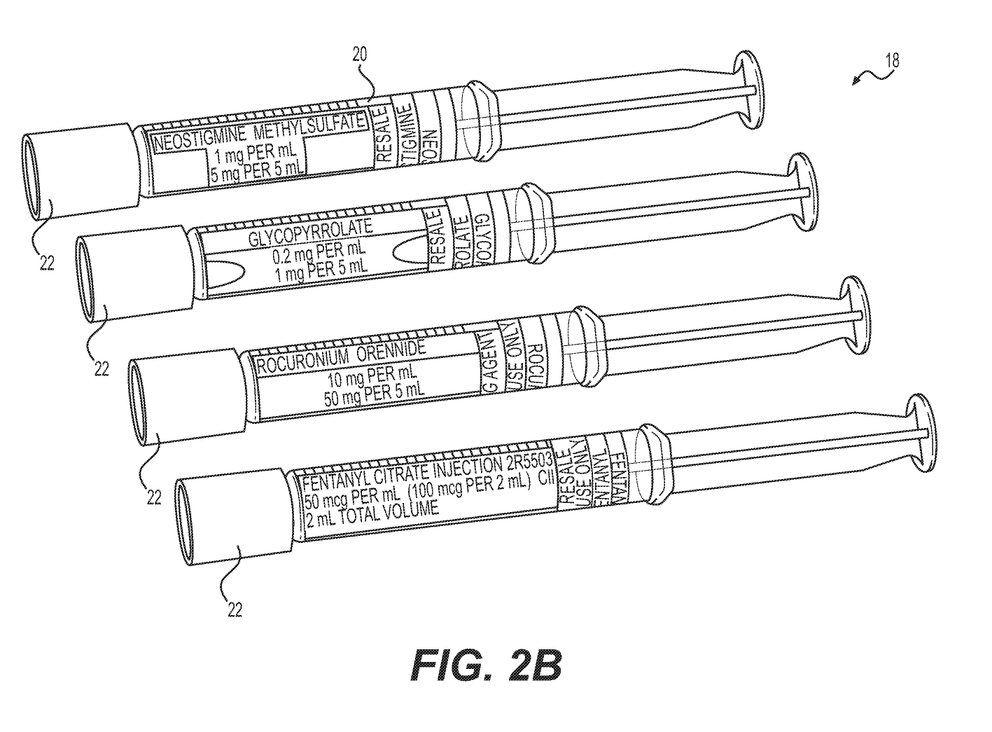

In order to produce the apparatus of the present disclosure, the cap and the vial portion of the apparatus are generally produced according to standard methods known in the art. An exemplary vial or tube of the present apparatus is a syringe. Accordingly, an illustrative embodiment of the present cap and vial portion of the present apparatus is a standard, capped syringe (see FIG. 2A). For example, a standard syringe is one used, typically by medical personnel, for storing, dispensing, and/or administering medications or medicaments to patients.

Starting from a standard, capped syringe, the one or more labels and/or sublabels of the present apparatus must be created. In addition, the one or more labels and/or sublabels must be affixed or attached to the cap and/or vial portion of the present apparatus. Creation of the one or more labels or sublabels of the present device comprises, consists essentially of, or consists of transcribing or imprinting on the surface of any material that may adhere to any portion of the present apparatus, particularly the vial and/or the cap portion of the apparatus. In addition, the labels or sublabels must be created such that the transcribed and/or imprinted language is shown on the label in a manner that is easily and quickly readable by a user, such as a medical personnel.

In addition, the labels and sublabels of the present apparatus may be created by transcribing, imprinting, and/or embedding of custom information using standard methods and techniques known in the art. For example, labels and sublabels of the present apparatus may be created by any printing method or mechanism, including but not limited to laser, digital, flexographic, three-dimensional (e.g., 3D), silk screen, ink jet, pad printing methods, stamping and hot stamping methods, thermal print, embossing, laser printing, shrink labeling methods, or by tying or clipping on a label that might act as a flag, and other such methods as known in the art. Further any printing apparatus or mechanism may be utilized to transcribe, imprint, and/or embed labels and/or sublabels of the present disclosure with relevant custom information about the medication or medicament, its proper use, and/or storage.

Once the label or sublabel is created, it may also be covered with a layer of a protective coating. The protective coating is added to the label to preserve and/or maintain the integrity of the information comprised on the label or sublabel. Any side or portion of the label or sublabel may be covered with the protective coating, particularly the front side of the label comprising the relevant information. In addition, the overcoat or protective coating may be added to the label or sublabel by any means that allows the coating to adhere to the label, including but not limited to spraying, dipping, drenching, etc.

Once the label and sublabels are created and/or coated, the labels and sublabels are then affixed or attached to any portion of the apparatus of the present disclosure. In particular, the labels and the sublabels of the present disclosure may be affixed or attached to the present apparatus by the adhesive component comprised on the label or the sublabel. In one embodiment, the adhesive component may be comprised on the back side of the label or the sublabel. In another embodiment, the label is embedded within an inner portion or compartment of a vial (e.g., a plunger, a plunger rod, and/or a plunger cap).

Accordingly, any portion of the label or sublabel may be brought into contact with any portion of the apparatus. More specifically, the back side of the label or the sublabel may be brought into contact with any portion of the present apparatus, such as the outer portion of the apparatus. In an illustrative embodiment, the back side of the label or sublabel may be brought into contact with the outer portion of the apparatus.

In another embodiment, the back side of the label or sublabel may be brought into contact and/or affixed, attached, or adhered to the outer portion of the cap of the present apparatus. In an additional embodiment, the back side of the label or sublabel may be brought into contact and/or affixed, attached, or adhered to the outer portion of the cap and/or the vial of the present apparatus. In an exemplary embodiment, the back side of the label or sublabel may be brought into contact and/or affixed, attached, or adhered to the outer portion of the cap and the vial of the present apparatus.

The labels and sublabels of the present apparatus may be affixed, attached, or adhered to the outer portion of the present apparatus at any time. More specifically, the labels and/or sublabels of the present apparatus may be affixed, attached, or adhered to the outer portion of the present apparatus before or after a medication or medicament is dispensed and/or delivered into the inner portion of the tube, the vial, or the syringe of the present apparatus. In addition, the labels and/or sublabels of the present apparatus may be affixed, attached, or adhered to the outer portion of the present apparatus before or after a cap of the present apparatus is brought into contact with the vial or tube portion of the present apparatus.

Likewise, a medication or medicament may be dispensed and/or delivered into the inner portion of the tube, the vial, or the syringe of the present apparatus at any time. More specifically, the medication or medicament comprised by the present apparatus may be dispensed or delivered into the inner portion of the tube or vial of the present apparatus before or after the apparatus has had the labels and/or sublabels attached or affixed thereto. In addition, a medication or medicament may be dispensed and/or delivered into the inner portion of the tube, the vial, or the syringe portion of the present apparatus before or after a cap of the present apparatus is brought into contact with the vial or tube portion of the present apparatus.

Once the label or sublabel is affixed, attached, or adhered to the outer portion and/or embedded within the inner portion of the cap and/or vial of the apparatus, and the medication or medicament has been dispensed and/or delivered into the inner portion of the tube, the vial, or the syringe portion of the present apparatus, the method of the present apparatus results in the production and/or formation of the present apparatus. In addition, the present apparatus is also readily available to a user, such as a medical personnel. Accordingly, the present disclosure also relates to a method of using the present apparatus.

The method of using the apparatus of the present disclosure may comprise a user. For example, the user of the present apparatus may be medically trained or untrained. In an illustrative embodiment, the user of the present apparatus is medically trained, such as a medical personnel (e.g., without limitation, a doctor, a physician, a physician's assistant, a nurse, an anesthesiologist, a nurse anesthetist, an emergency medical technician or EMT, emergency and operating room personnel, etc.). In addition, the user of the present apparatus employs the present apparatus, such as in a medical or veterinary environment, in order to safely improve identification, selection, disbursement, distribution, and administration of medications and medicaments to patients.

More specifically, in order to properly utilize the present apparatus, the user may visually or physically inspect the apparatus for the one or more labels or sublabels affixed or attached thereto. For example a user may visualize, inspect, read, and/or identify the labels on the vial or the cap of the present apparatus. In particular, the colors, codes, (e.g., barcodes), and information contained or maintained on the label or sublabel of the apparatus should be easy, clear, and quickly interpreted and/or deciphered by a user to indicate the type and dose of medication or medicament comprised therein.

Text and/or lettering on the label of the present apparatus may be in any size, font, orientation, boldness, etc. in order to aid differentiation between apparatuses comprising different drugs medications and/or medicaments within the same drug classes. In addition, since the surface area on the vial and cap of the present apparatus to which a label may be affixed and/or attached is typically very limited, labels primarily comprising, consisting essentially of, or consisting of the ASTM color codes and/or codes (e.g., barcodes) provide a much easier and efficient mechanism to visualize, identify, and/or read the proper type, dose, concentration, and/or volume of medication or medicament comprised therein.

Alternatively, the labels on the present apparatus, such as the labels on the vial or the cap of the present apparatus, may be manually, automatically, digitally, and/or electronically scanned by any type of a scanning device that is able to read and/or decipher the information transcribed, imprinted, and/or embedded within the label. For example, a label of the cap or vial of the present apparatus may be digitally scanned by a scanning device that includes, but is not limited to a computer, an automated device, a stationary device, a mobile, portable, and/or handheld device, or any type of a code reader (e.g., a barcode reader and/or a color code reader). In an illustrative embodiment, a barcode on the label of the vial or cap may be digitally scanned, read, and/or translated by the scanning device that may include, but is not limited to a computer, an automated device, a stationary device, a mobile, portable, and/or handheld device, and/or any type of a barcode reader, and/or any type of visual recognition scanning device, and/or any type of RFID reader, and/or any type of remote electronic detection device.

Further, the present disclosure relates to a method of storing, retrieving, and/or dispensing a medication or medicament to a patient. More specifically, the method of the present disclosure relates to storing, selecting, and or dispensing prescription drug medications and medicaments from a dispensary compartment to a patient. Illustrative dispensary compartments of the present disclosure include, but are not limited to drawers, cabinets, refrigerators, freezers, and/or any standard compartment known or used in the art that is capable of properly storing drug medications and medicaments. Medications and medicaments comprised in the present apparatus may be further stored in a dispensary compartment until retrieved for identification, selection, distribution, and/or administration to a patient.

The method of the present disclosure relates to storing, retrieving, and/or dispensing medications and medicaments comprised within the present apparatus in a dispensary compartment. For example, in the present method, the apparatus of the present disclosure has the capability of being stored in a vertical position within a dispensary compartment, such as a drawer or a cabinet. In addition, a user or a scanning device may easily identify, select, distribute, and/or administer medications or medicaments that are vertically stored in the dispensary drawer by translating and/or deciphering the information comprised in the label attached to, affixed to, and/or embedded within the cap of the apparatus.

While the present apparatus may be vertically stored within the dispensary compartment with the cap positioned upward or downward, typically, the apparatus will be positioned such that the cap is positioned upward nearest to the user and/or scanning device. In certain embodiments, during storage in a dispensary compartment, the present apparatus is positioned vertically, such that the label on the cap of the apparatus is visible, accessible, and/or decipherable to a user and/or a scanning device (as described above). Once vertically positioned, the label of the cap of the apparatus may be identified and/or visualized by the user and/or scanning device in order to enable accurate and easy selection, disbursement, distribution, and/or administration of the specific medication and medicament to a patient. In one embodied example permitting improved identification, syringes would be stored vertically with a plunger rod up, such that users could see the plunger rod color to differentiate the different drug classes and/or drugs.

Importantly, the method of vertically storing, retrieving, and/or dispensing medications and medicaments comprised in the present apparatus as described herein provides a benefit over existing technologies. More specifically, existing technologies and methods generally require vials and syringes comprising medications and medicaments to be stored horizontally in order for a label comprised on the body of the vial to be visible, accessible, and/or decipherable to a user and/or a scanning device. However, the present method comprising, consisting essentially of, or consisting of vertical positioning of the present apparatus within a dispensary compartment allows much more accurate, efficient, and economical storage, retrieval, and disbursement of medications and medicaments to a patient. These results were surprising and unexpected.

In further embodiments, an improved horizontal storage is further contemplated over existing technologies. As an example, certain containers may be stored horizontally rather than vertically such as syringes or IV bags. The present invention is advantageous for such horizontal storage because labeling, patterns, color schemes and other identifiable features described here permit improved identification of the drug class, or of the specific medicament or concentration. In particular, the caps of a syringe and/or an IV bag would typically be labeled with ASTM coloring, and/or a label containing the name of the drug, and so would permit a healthcare professional to quickly and accurately identify a drug class or a drug, including based on the context or circumstances of the storage and use. Similarly, other containers having a cap with ASTM coloring and/or a label containing the name of the drug could also advantageously be stored horizontally if otherwise permitted by health authorities and the manufacturer. In one example embodiment of the present invention, containers such as syringes may be stored horizontally. When stored horizontally it is easy for a user to read the syringe labels, and syringe caps. They are easy to distinguish side by side from one another, first by the drug class on the syringe cap color, and additionally by the name of the drug on the syringe cap, which can be seen from any placement when horizontal. Typically, syringe labels will be a different design from each different drug family, with an additional different color used for each different strength.

Text and/or lettering on the label of the present apparatus may be in any size, font, orientation, boldness, etc. in order to aid differentiation between apparatuses comprising different drugs medications and/or medicaments within the same drug classes. Notably, the colors, codes, shapes, text and/or lettering comprised on the label or sublabel of the present apparatus will enable quicker and faster identification, selection, distribution, and administration of medications and medicaments to patients that results in increased safety of those patients. In particular, the colors, design, and/or lettering coded on the cap and/or the vial of the present apparatus will enable quicker and faster identification, selection, distribution, and administration of medications and medicaments to patients that further results in increased safety of those patients. Importantly, the colors, design, and/or lettering coded on the cap of the present apparatus will enable quicker and faster identification, selection, distribution, and administration of medications and medicaments to patients that results in increased safety of those patients.

### EXAMPLES

Illustrative embodiments of the apparatus and methods of the present disclosure are provided herein by way of examples. While the concepts and technology of the present disclosure are susceptible to broad application, various modifications, and alternative forms, specific embodiments will be described here in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

### Example 1

### Existing Technology

Existing technologies to improve the ease of identification, selection, distribution, and administration of drug medications to patients begins with modification of a standard, unlabeled vial (i.e., a syringe) 18 comprising a white, unlabeled syringe cap 22 (see FIG. 2A). Improvements in the existing technology used in the medical industry have lead manufacturers to label a medicinal vial (i.e., a syringe 18 or container) with the ASTM color code. As shown in FIG. 2B, the body of existing syringes 18 may be labeled with the ASTM color code indicating the ASTM drug class, the drug name, and/or the drug dose associated with illustrative medications, such as Neostigmine (i.e., red and white diagonal stripe), Glycopyrrolate i.e., green), Rocuronium (i.e., red), and Fentanyl (i.e., light blue) on the respective syringe bodies 20. However, the caps 22 on these prior art syringes 18 are not labeled with the ASTM color code indicating the ASTM drug class, the drug name, and/or the drug dose at all (as shown in FIGS. 2A and 2B). Alternatively, the white caps 22 on existing syringes are labeled with a manufacturer's brand and/or logo.

### Example 2

### The Vial of the Present Apparatus

The present apparatus comprises a vial (e.g., a syringe 18) and a cap 22, wherein the cap 22 comprises a label. One embodiment of the present apparatus comprises a vial and a cap, wherein both the vial and the cap each comprise a label. An exemplary embodiment of the present apparatus comprises a cap 22 and a corresponding vial (i.e., a syringe 18) of the present disclosure both comprising a label indicating the ASTM color (i.e., green) and the drug name (i.e., Glycopyrrolate). In typical embodiments, each concentration of dosage has its own packaging color to identify each different strength within the same drug family.

More specifically, the cap 22 of the apparatus embodiment shown in FIG. 3A comprises a label 24 that is colored green to represent the ASTM drug class of anticholinergic agents. In particular, the label 24 of the cap 22 is further transcribed to show the drug name “Glycopyrrolate” in both black and white diagonal lettering. The cap 22 may be attached or affixed to the vial portion of the present apparatus (i.e., the syringe body 20), which also comprises a label.

For example, on this embodiment of the present apparatus (see FIG. 3A, FIG. 3B), a label 26 affixed to the syringe body portion 20 of the apparatus comprises a first sublabel 27 indicating the total drug content (0.6 mg), the drug volume (3 ml), and the final drug dose (i.e., 0.2 mg per ml). A label attached to the syringe portion 26 of the apparatus also comprises the name of the medicament and may have a color or pattern configuration based on the total drug content, drug volume, or final drug dose or any other attribute. In the embodiment of FIG. 3A, each of the three syringes 18 and labels 26 would typically have different colors based on drug content (including, e.g., a different ASTM color typically indicating a drug class), volume and/or dose, while a separate syringe body label 30 on each syringe 18 would have a common color and/or pattern configuration as the cap 22 and/or cap label 24 (including, e.g., a different ASTM color typically indicating a drug class). In a typical embodiment the syringe body label 30 for this drug would be green. The label 26 and/or syringe body label 30 may additionally comprise a second sublabel 28 that indicates the volume of the syringe (i.e., 3 ml), as well as the National Drug Code (NDC; e.g., NDC 70860-XXX-00) (not all shown in FIG. 3A or FIG. 3B). Further information that may not be shown in FIG. 3A, but may be transcribed on the label 26, first sublabel 27, and/or second sublabel 28 shown in FIG. 3A includes: 1) the express volume of the syringe (e.g., 5 ml), 2) the pharmacy from which the drug was obtained (e.g., Rₓ), 3) storage conditions (e.g., room temperature storage, avoid freezing, protect from light, etc.), 4) lot number, and 5) expiration date of the drug, as well as 6) the syringe manufacturer (e.g., Athenex). Finally, the syringe body 20 label embodiment 26-28 comprises space for an optional barcode that comprises, consists essentially of, or consists of any scannable or electronically readable information about the medication or medicament that is deemed necessary by a user or manufacturer of the present apparatus. Additionally, multiple different sublabels may exist within or adjacent to or otherwise affixed to the syringe body 20.

### Example 3

### The Cap of the Present Apparatus

The present apparatus comprises a vial (e.g., a syringe 18) and a cap 22, wherein the cap comprises a label 24. Caps 22 of the present disclosure may be formatted or transcribed with a label comprising any information and with any design, color, or lettering so as to easily identify the type or dose of drug comprised within the apparatus. Five embodiments of caps 22 of the present apparatus are shown in FIG. 3C. The caps 22 are formed to attach or affix to the vial portion of the apparatus of the present disclosure (e.g., a syringe 18) as shown in FIGS. 3A, 3B. It is understood from the disclosure that a cartridge and auto-injector are also included as similar embodiments to the above, whether expressly disclosed in a figure or example or not, because cartridges and/or autoinjectors are commonly used like syringes. In typical embodiments of FIGS. 3A and 3B, each concentration of dosage has its own packaging color to identify each different strength within the same drug family.

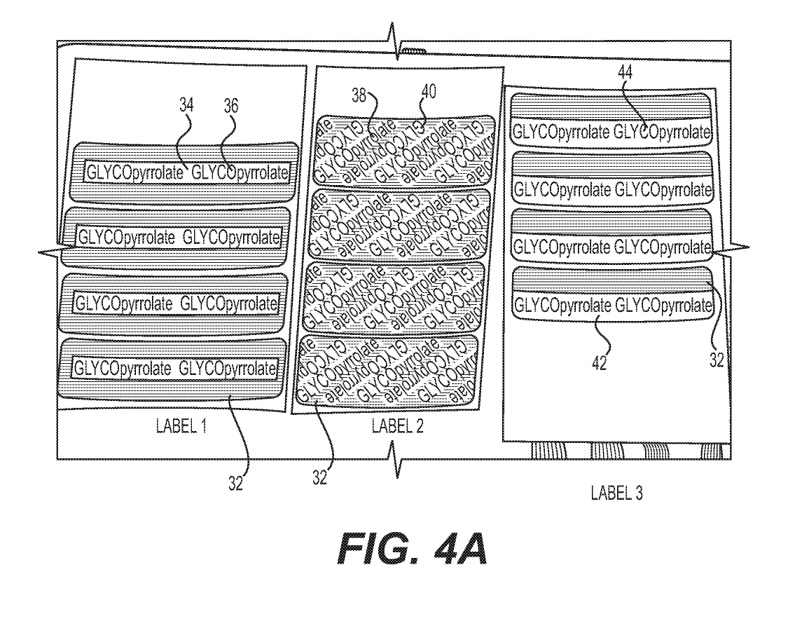

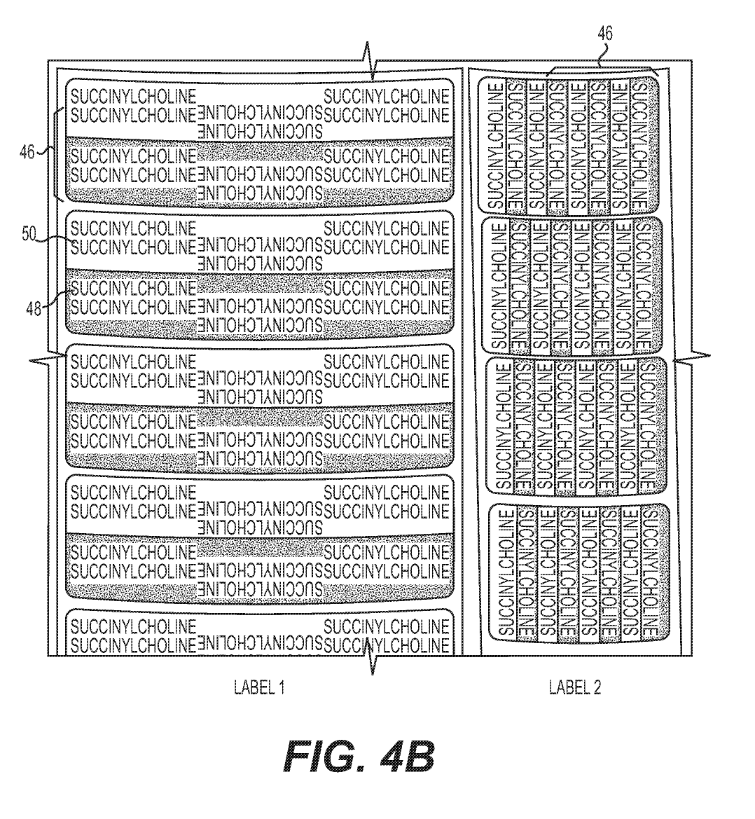

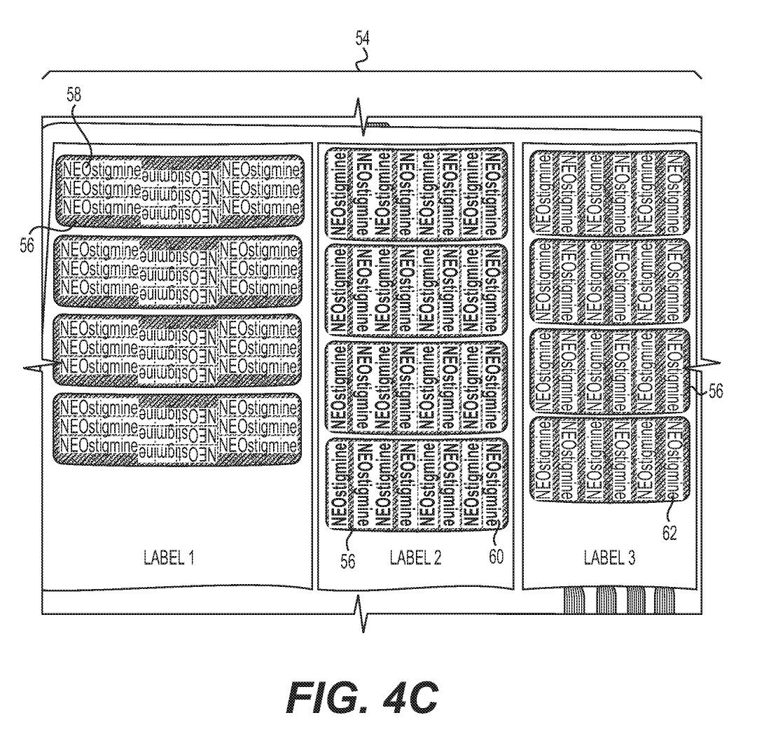

The illustrative caps 22 of FIG. 3C in particular are labeled with different designs comprising the ASTM color and the drug name; reference numerals are cross-identified in other figures. For example, 1) Succinylcholine (i.e., caps 1 and 2 comprise red and black colors and horizontal and vertical lettering in red and black) (labels 1 and 2 of FIG. 4B), 2) Neostigmine (i.e., Caps 3 and 4 comprise red and white diagonal stripes with horizontal and vertical lettering in black and white) (FIG. 4C), and 3) Glycopyrrolate (i.e., cap 5 comprises green and white colors with black horizontal lettering) (FIG. 4A). In certain other embodiments of caps 22 (not shown in FIG. 3C), for products that use a fluorescent red ASTM, or where there are numerous drugs that use the same or similar color, then certain other embodiments of caps 22 would have a different cap design, and/or color, and/or label, and/or pattern, for each drug in that class using the florescent red ASTM color as the primary color. Thus, improved identification of drugs would be achieved. Similarly, where there would otherwise be confusion or less than optimal identification or differentiation of products based on ASTM or other primary color, cap 22 label design, including color and/or patterns, and/or text would be used to differentiate each drug within a class, or to differentiate drugs between in different classes in certain cases.

### Example 4

### The Labels of the Present Apparatus

The present apparatus comprises a vial (e.g., a syringe 18) and a cap 22, wherein the cap 22 comprises a label 24. Labels of the present disclosure may be transcribed, imprinted, or formatted with any information and with any design, color, or lettering so as to easily identify the type or dose of drug comprised within the apparatus. Three embodiments of labels for caps 24 of the present apparatus are shown in FIG. 4A. The caps 22 are formed to attach to or affix to the vial portion of the apparatus of the present disclosure (e.g., a syringe 18) as shown in FIG. 3A.

Each label embodiment is transcribed, imprinted, or comprises a different variation or different designs comprising the ASTM color (green) 32, along with horizontal or diagonal black and white lettering of the drug name. For example, labels 1 of FIG. 4A comprise a green background color 32 with a white, center block 34 comprising horizontal lettering of the drug name (“Glycopyrrolate”) in black 36. Labels 2 comprise a green background color 32 comprising alternating, diagonal lettering of the drug name (“Glycopyrrolate”) in black 38 and white 40 (see FIG. 4A). Labels 3 comprise a green background color 32 with a white, bottom stripe 42 comprising horizontal lettering of the drug name (“Glycopyrrolate”) in black 44 (see FIG. 4A). Typically, “LABEL 2” diagonal configuration would be used on caps in commercial products of glycopyrrolate, although the invention contemplates commercial use of all of the labels.

In addition, labels 1 and 2 of FIG. 4B comprise an alternating red and black block striped background 46. Labels 1 of FIG. 4B comprises double, alternating black lettering of the drug name (“Succinylcholine”) on the red block background 48 and double, alternating red lettering of the drug name (“Succinylcholine”) on the black block background 50. Labels 2 comprise a single recitation of black lettering of the drug name (“Succinylcholine”) on the red block background 48 and a single recitation of red lettering of the drug name (“Succinylcholine”) on the black block background 50 (see FIG. 4B).

Finally, each label embodiment of FIG. 4C is transcribed, imprinted, or comprises a different variation or different designs comprising the ASTM color, i.e., red and white diagonal stripes 56, along with horizontal or diagonal black and white lettering of the drug name 58, 60, 62, Neostigmine, i.e., labels 1-3 54. For example, labels 1 of FIG. 4A comprise a red and white diagonal striped background 56 with a triple, alternating black and white horizontal lettering of the drug name (“Neostigmine”) in a particular font 58. Labels 2 comprise a red and white diagonal striped background comprising alternating, vertical lettering of the drug name (“Neostigmine”) in black and white in particular font 60 (see FIG. 4C). Labels 3 comprise a red and white diagonal striped background with large alternating, vertical lettering of the drug name (“Neostigmine”) in black and white in a particular font 62 (see FIG. 4C). The particular fonts chosen for each label may be the same as, or differing than other labels in certain embodiments.

### Example 5

### The Labels and Sublabels of the Present Apparatus

The labels of the present apparatus may further comprise one or more, two or more or a plurality of sublabels. In particular the label for the vial portion of the present apparatus (e.g., a syringe) may comprise one or more, two or more or a plurality of sublabels. More specifically, FIG. 5A is a schematic of an embodiment of a label for a syringe 64, with corresponding cap 22 (shown in FIGS. 3A and 5C), transcribed, imprinted, or comprising the ASTM color (i.e., red and white diagonal stripes) 66 indicating the drug class relaxant antagonist (non-depolarizer). The label further comprises the drug name, Neostigmine, in horizontal white lettering 68, and also in black lettering 70. In addition, the illustrative label of FIG. 5A comprises a first and a second sublabel.

Here, the illustrative first sublabel 72 has been transcribed or imprinted with the total drug content (5 mg), the drug volume (5 ml), and the final drug dose (i.e., 1 mg per ml) 74 that is also typically indicated by the full label (not shown). In addition, the specific concentration and/or volume of Neostigmine (i.e., 5 mg per 5 ml) 74 are indicated on the first sublabel 72 located at the top of the syringe label. Further, the first sublabel 72 highlights the drug name, the drug dose, and/or the drug concentration with an additional color for easy identification. For example, in the illustrative embodiment of FIG. 5A, the color around the drug name 68 is blue 76. The blue color around the drug name 76 in the first sublabel 72 indicates the specific drug as indicated by the drug name (i.e., Neostigmine), the drug volume (i.e., 5 ml), and/or or the drug dose (i.e., 1 mg per ml).

As shown on FIG. 5A, the blue color around the drug name 76 on the first sublabel 72 may be further outlined by the background color indicating the drug class. For example, the red and white diagonal stripes 66 indicating the relaxant antagonist drug class around the blue color 76 highlighting the drug name. Typically, this drug class identifier would be an ASTM color, or related to the same. This additional color code on the first sublabel helps further identify, select, distribute, and administer the proper type and/or dose of medication or medicament to a patient.

This label embodiment further comprises a second sublabel 78 typically located below the first sublabel 72 (see FIG. 5A). The second sublabel 78 of this syringe label embodiment further comprises, consists essentially of, or consists of the following information as indicated in FIG. 5A related to the drug medication or medicament comprised therein: 1) National Drug Code (NDC; e.g., NDC 70860-XXX-00) 80, 2) the volume of the syringe (e.g., 5 ml) 82, 3) the pharmacy from which the drug was obtained (e.g., Rₓ) 84, 4) storage conditions (e.g., room temperature storage, avoid freezing, protect from light, etc.) 86, 5) lot number 88, 6) expiration date of the drug 90, as well as 7) the syringe manufacturer (e.g., Athenex) 92. Finally, the label embodiment comprises space for an optional barcode 94 that comprises, consists essentially of, or consists of any scannable or electronically readable information about the medication or medicament that is deemed necessary by a user or manufacturer of the present apparatus. Typically, this information 80-94 on the second sublabel 78 would be placed generally in the positions indicated in FIG. 5A, but variations in text size, location, font style, usage of capitals or non-capitals may be employed for easier or more convenient access or identification in stocking, logistics, or use of medicament containers.

FIG. 5B is a schematic of four embodiments of a label for a syringe, with corresponding cap 22 (see FIGS. 3A and 5C), comprising an ASTM color (red and white diagonal stripes) 96, along with vertical or horizontal black and red lettering of the drug name 98, Neostigmine. In addition, these label embodiments comprise all of the information specified in FIG. 5A above, which may be transcribed on a first and/or a second sublabel.

In one embodiment of the label of the present disclosure, a first sublabel is colored blue around the drug name 100, and comprises the total drug content (5 mg), the drug volume (5 ml), and the final drug dose (i.e., 1 mg per ml) as shown in more detail in FIG. 5A. In a second embodiment (i.e., label 2), the first sublabel is colored red around the drug name 102, and comprises the total drug content (4 mg), the drug volume (4 ml), and the final drug dose (i.e., 1 mg per ml). A third embodiment of the first sublabel (i.e., label 3) of FIG. 5B is colored yellow 104 around the drug name, and comprises the total drug content (3 mg), the drug volume (3 ml), and the final drug dose (i.e., 1 mg per ml). A final sublabel embodiment exemplified herein (i.e., label 4) comprises the first sublabel that is colored green around the drug name 106, and comprises the total drug content (2 mg), the drug volume (2 ml), and the final drug dose (i.e., 1 mg per ml).

Each of the syringe label embodiments of FIG. 5B comprise a second sublabel 108, 110 located below the first sublabel (not shown for labels 1 and 3, but shown for labels 2 and 4). The second sublabel further comprises, consists essentially, or consists of the following information related to the drug medication or medicament comprised therein: 1) National Drug Code (NDC; e.g., NDC 70860-XXX-00), 2) the volume of the syringe (e.g., 5 ml), 3) the pharmacy from which the drug was obtained (e.g., Rₓ), 4) storage conditions (e.g., room temperature storage, avoid freezing, protect from light, etc.), 5) lot number, 6) expiration date of the drug, as well as 7) the syringe manufacturer (e.g., Athenex). Finally, the label embodiment comprises space for an optional barcode that comprises, consists essentially of, or consists of any scannable or electronically readable information about the medication or medicament that is deemed necessary by a user or manufacturer of the present apparatus.

### Example 6

### The Present Apparatus

FIG. 5C is a figure showing an embodiment of the apparatus of the present disclosure comprising a cap 22 and a corresponding syringe body 20 both labeled with an ASTM color (i.e., red and white diagonal strips) 112 and the drug name (i.e., Neostigmine). This embodiment of the present apparatus comprises a label on the cap 24 that further comprises alternating, horizontal lettering in white and black for safe identification, selection, distribution, and administration of the medication and medicaments comprised therein.

The label embodiment of the vial of the present apparatus shown in FIG. 5C comprises a first sublabel and/or a second sublabel (not shown). The first sublabel of the illustrative embodiment is colored orange around the drug name, Neostigmine. In addition the first sublabel indicates the total drug content (1 mg), the drug volume (1 ml), and the final drug dose (i.e., 1 mg per ml) comprised by apparatus (see FIG. 5C). The second sublabel (not shown) may further comprise, consist essentially of, or consist of the following information related to the drug medication or medicament comprised therein: 1) National Drug Code (NDC; e.g., NDC 70860-XXX-00), 2) the volume of the syringe, 3) the pharmacy from which the drug was obtained, 4) storage conditions, 5) lot number, 6) expiration date of the drug, as well as 7) the syringe manufacturer. In typical embodiments, the cap contains ASTM colors, as does at least portion of the label which helps to identify the drug class when the cap is removed during use.

### Example 7

### The IV Bag Apparatus

FIGS. 6A-6D are diagrams of embodiments of an IV bag apparatus, and sets thereof. FIGS. 6A-6D indicate apparatuses that may comprise certain sets of IV bags that can be stored, used, or shipped (as disclosed above and herein). Certain of the IV bags have the same active pharmaceutical ingredient but a different dosage, which in turn informs the color, text, and/or label scheme or configuration to allow easy visibility. As discussed herein, and as indicated in FIG. 6, certain configurations of ASTM colors may be selected for a particular drug or medicament class, and certain other colors, labeling, and text may be selected for dosages in an easily visible fashion given the typical usage of an IV bag for patient treatment. In the particular embodiments indicated in FIGS. 6A-6D, port color configuration whether medicament or IV tube 112, 114 is selected to quickly convey additional information to a healthcare provider or assistant. Typically, the port color information would match the ASTM configuration for a particular drug class so that the time of physically beginning to administer by the medicament by opening the port a final verification may be conducted to prevent human error with respect to drug class. In other embodiments, both ports, i.e., the medicament port and the IV tubing port 112, 114, may be selected with the same color configuration, or with a different color configuration. In this manner, multiple different meanings may be quickly conveyed to healthcare providers or assistants. The embodiments of FIG. 6 further illustrate the diversity of color configurations allowed. When multiple dosage forms exist or are used, the packaging, label, and color patterns therein may be adjusted to portray a plurality of different dosages or other information about a medicament. As an example, the FIG. 6 phenylephrine IV bags are embodied in five different bags, each with varying label and color configurations. Further, in FIG. 6, the norepinephrine and epinephrine are embodied with two different bags, which in some cases may reduce the type of required color configurations needed for quick visibility in a logistics or healthcare provider setting.

The embodiment in FIG. 6A contains a label 116 on the container, i.e., an IV bag. Typically, the label background color is white, but other colors and patterns are contemplated in the invention herein. The labels of FIG. 6A contain at least three separate color configurations in this particular embodiment. First, a stripe that could be fading into a label background, and/or with a pattern appears on the left side 118 of the label. Second, a stripe that could be fading into a label background and/or with a pattern appears on the right side 120 of the label. Third, a pattern or shape, here a representation of a triangle, is identified on the label containing the number of units of the medicament in the bag 122. The stripes 118, 120 may appear to be passing under the triangular shape 122 with a small portion outside the other end 124. Configurations of the medicament port 112 are contemplated to align with these color, shape, and/or pattern configurations 116-124 as discussed herein for greater, safer, and more efficient ease of usage in terms of medical, stocking, or logistics. Additionally, the text of the drug name 126 would typically be on the IV bag and may contain varying font style, size, case, and also may have a separate color background corresponding to other indications or configurations on the label 116 or other packaging (not shown).

FIG. 6B illustrates a further embodiment of the IV bag invention. Here, a label 116 contains identifying color and/or pattern configurations. A first color and/or pattern of the IV bag label 128 in this embodiment matches the color of the text of the drug name 130 and the total drug content 132, i.e., 4 mg and 8 mg. This color 128 would be configured based on the drug concentration in the IV bag, and thus BAG 1 would have a different color 128 than BAG 2. A second color and/or pattern 134 would typically be the same for each of BAG 1 and BAG 2 because the second color is typically configured by the type of drug or medicament which in FIG. 6B is the same (typically the second color and/or pattern 134 would be the ASTM color indicating drug class). As discussed herein, the port cap of FIG. 6B may also be configured to correspond with other color or information on the IV bag or label 116.

FIG. 6C illustrates a further embodiment of the IV bag invention. Here, a label 116 on each different bag contains multiple different color and/or pattern configurations. Some of these patterns described below are embodied in an angled configuration. In a typical embodiment the portion 136 is a certain color and/or pattern that would typically correspond to the total medicament or drug content and/or concentration in the IV bag. The portion 138 is a certain color and/or pattern typically different than the first color portion 136. The second color portion 138 is many times a lighter shade than the first portion 136, but this is not always the case and it may or may not be matching. Generally, a first portion of the text of the drug of medicament is in capital letters 139 that corresponds to the same color as the second color portion 138 for improved visibility, identification, and verification. The remainder of the text of the drug is typically white but other color configuration for improved visibility, identification, and verification may be selected as described herein. Generally, the text of the total drug content and the solution 137 is the same color as the first color portion 136 although in certain configurations when the colors could clash or visibility or identification may be reduced then such colors may not be exactly the same color or configuration. A further color and/or pattern portion 140 is indicated on the label 116. In FIG. 6C, this particular color portion 140 is indicated by diagonal stripes of two lighter shades of either one or typically both of the first and second color portions 136, 138. Generally, a color bar 142 will be black surrounding the solution identification but other colors are contemplated. The color portion 134 typically near the bottom of the label 116 is the same color for all of the bags in FIG. 6C because they contain the same drug/medicament. The lettering of the drug in this portion 134 is generally white, but other colors may be selected for improved visibility, identification, and verification. However, it is contemplated that each of the color portions, bars, diagonals, pattern(s), text, font, size, or portions thereof may be configured to correspond to any of the information or other colors/patterns or other configurations indicated on the bags or on the IV labels. As discussed herein, the port cap 112 and/or IV cap 114 of FIG. 6C may also be configured to correspond with other color or information on the IV bag or label 116.

FIG. 6D illustrates two embodiments of IV bags, i.e., BAG 1 and BAG 2 with labels 116. Each bag has a first color portion 144 on the label typically corresponding to the total drug/medicament content in the bag. For example, the first color portion 144 of BAG 1 or BAG 2 would be visibly different, and may be, e.g., a bright orange for BAG 1 and a purple for BAG 2. A color portion 146 surrounding the drug/medicament total content number is also present in the label 116. In both bags this may be a color such as yellow. Typically, a third color portion 150 would match the color portion surrounding the total content 146. In this particular embodiment, both color portions 146, 150 are the same as each other and the same between BAG 1 and BAG 2, e.g., since they are the same type of drug, but other configurations and permutations for improved visibility are contemplated. A fourth portion 152 indicates a changing color and/or pattern designation between the color portion indicating the total drug content 144 and the lower color portion 150. Generally, the text of the solution 154 and the text of the total content of the bag 156 would be expected to be the same as the background drug content color 146 (and typically also the lower portion color 150). However, this text 154, 156 need not be the same such color and different configurations are contemplated. The color portion 134 typically near the bottom of the label 116 is the same color for the bags in FIG. 6D because they contain the same drug/medicament. The lettering of the drug in this portion 134 is generally white, but other colors may be selected for improved visibility, identification, and verification. A concentration is indicated near the bottom of the label 116 with a particular corresponding color and/or pattern configuration 148. Additionally, the text of the concentration is indicated in FIG. 6D in at least two places 158, 160. This text 158, 160 is typically white, but other configurations, including differences in the text 158, 160 in different locations on the label 116 are contemplated for improved visibility. As discussed herein, the port cap 112 and/or IV cap 114 of FIG. 6C may also be configured to correspond with other color or information on the IV bag or label 116.

Typically, it is understood that when practicing the invention ASTM colors for IV bags would be utilized 134 to indicate a base color of the label to identify the drug class (FIGS. 6B, 6C, 6D). As discussed above, and as is typically present in the other embodiments of the invention, the containers would further have a unique label design for each drug, with a unique color combination for each strength of drug within each drug family. However, with regards to FIG. 6A, vasopressin does not have an ASTM color. It is noted that prior art vasopressors typically have the same packaging and may have names sounding alike. Thus the embodiments of the present invention provide distinguishments for different aspects of vasopressors for improved recognition. However, for those drugs that do have an ASTM color, e.g., without limitation, norepinephrine, an ASTM color is typically added to the container to indicate drug class.

Although not necessarily depicted, the labels 116 of the IV bags in FIGS. 6A-6D would typically also be contemplated to contain other information discussed herein, e.g., bar code information, manufacturer, prescription, dosage, total volume, total drug content, concentration, storage conditions, lot, and expiration. Color, text, pattern or other schemes or configurations described herein may also correspond to any such other information on the container. In typical embodiments, each concentration of dosage has its own packaging color to identify each different strength within the same drug family. Further, in typical embodiments, the same copy of a label on an IV bag would be on the both sides of the bag so that it would be visible when hanging from an IV pole.

The preceding description enables others skilled in the art to utilize the technology in various embodiments and with various modifications as are suited to the particular use contemplated. In accordance with the provisions of the patent statutes, the principles and modes of operation of this disclosure have been explained and illustrated in exemplary embodiments. Accordingly, the present invention is not limited to the particular embodiments described and/or exemplified herein.

It is intended that the scope of disclosure of the present technology be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims. The term “e.g.” or “for example” even if not expressly identified as providing example or alternative embodiments of the invention without limitation should be understood in all instances to be providing to a POSA example or alternative embodiments of the invention without limitation to other examples or alternative embodiments of the invention.

The scope of this disclosure should be determined, not only with reference to the above description, but should instead be determined with reference to the appended claims, along with the full scope of equivalents to which such claims are entitled. It is anticipated and intended that future developments will occur in the arts discussed herein, and that the disclosed compositions and methods will be incorporated into such future examples.

Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the disclosure and that the technology within the scope of these claims and their equivalents be covered thereby. In sum, it should be understood that the disclosure is capable of modification and variation and is limited only by the following claims.

We claim:

1. An apparatus for improving administration of a medication or medicament to a patient, the apparatus comprising:
a. a container,
b. a cap, and
c. one or more labels, wherein the cap of the apparatus comprises a label.

2. The apparatus of claim 1, wherein the container of the apparatus comprises a label.

3. The apparatus of claim 2, wherein the label is affixed to the outer portion of the cap or the outer portion of the container of the apparatus.

4. The apparatus of claim 2, wherein the label is affixed to the outer portion of the cap and the outer portion of the container of the apparatus.

5. The apparatus of claim 1, wherein the inner portion of the container further comprises the medication or medicament.

6. The apparatus of claim 5, wherein the label comprises a color code.

7. The apparatus of claim 6, wherein the color code on the label corresponds to the ASTM drug class to which the medication or medicament belongs.

8. The apparatus of claim 6, wherein the color code on the label corresponds to the drug volume or the drug dose of the medication or medicament comprised therein.

9. The apparatus of claim 1, wherein the label comprises information selected from the group consisting of one or more color codes, the total drug content, the drug volume, the final drug dose, the National Drug Code, the volume of the container, the pharmacy from which the drug was obtained, storage conditions, lot number, expiration date of the drug, and the name and/or location of the container manufacturer.

10. The apparatus of claim 1, wherein the label comprises one or more sublabels.

11. The apparatus of claim 10, wherein one or more sublabels comprise a first sublabel.

12. The apparatus of claim 10, wherein the one or more sublabels comprise a second sublabel.

13. The apparatus of claim 10, wherein the one or more sublabels comprise a first sublabel and a second sublabel.

14. The apparatus of claim 13, wherein the first sublabel and the second sublabel comprise information selected from the group consisting of one or more color codes, the total drug content, the drug volume, the final drug dose, the National Drug Code, the volume of the container, the pharmacy from which the drug was obtained, storage conditions, lot number, expiration date of the drug, and the name and/or location of the container manufacturer.

15. The apparatus of claim 1, wherein the cap further comprises a tamper evident cap, a tamper evident seal, or a tamper evident label.

16. The apparatus of claim 1, wherein the container further comprises a plunger rod.

17. The apparatus of claim 16, wherein the plunger rod comprises a label.

18. The apparatus of claim 17, wherein the label is internally embedded within the plunger rod.

19. The apparatus of claim 18, wherein the label comprises information selected from the group consisting of one or more color codes, the total drug content, the drug volume, the final drug dose, the National Drug Code, the volume of the container, the pharmacy from which the drug was obtained, storage conditions, lot number, expiration date of the drug, and the name and/or location of the container manufacturer.

20. The apparatus of claim 19, wherein the label comprises a color code.

21. The apparatus of claim 1, wherein the apparatus improves the safe identification, selection, distribution, and administration of medications and medicaments to patients.

22. A method of producing the apparatus of claim 1, wherein the method comprises:
imprinting colors, designs, or lettering on the label, and
attaching the label to the cap or the container of the apparatus.

23. The method of claim 22, wherein the label is attached to the outer portion of the cap or the outer portion of the container of the apparatus.

24. The method of claim 22, wherein the label is attached to the outer portion of the cap and the outer portion of the container of the apparatus.

25. The method of claim 22, wherein the inner portion of the container further comprises the medication or medicament.

26. A method of using the apparatus of claim 1 by a user, wherein the method comprises:
visualizing the colors, codes, designs, or lettering on the label of the cap, wherein the colors, codes, designs, or lettering on the label of the cap improve identification, selection, distribution, and administration of medications and medicaments to patients.

27. A method of dispensing the apparatus of claim 1 by a scanning device, wherein the method comprises:
vertically or horizontally positioning the apparatus in a dispensary compartment,
reading the colors, codes, designs, or lettering on the label of the cap,
wherein the colors, codes, designs, or lettering on the label of the cap improve identification, selection, distribution, and administration of medications and medicaments to patients,
retrieving the apparatus from the dispensary compartment, and
dispensing the apparatus for administration to a patient.

28. A method of dispensing the apparatus of claim 1 by a user, wherein the method comprises:
vertically or horizontally positioning the apparatus in a dispensary compartment,
reading the colors, codes, designs, or lettering on the label of the cap,
wherein the colors, codes, designs, or lettering on the label of the cap improve identification, selection, distribution, and administration of medications and medicaments to patients,
retrieving the apparatus from the dispensary compartment, and
dispensing the apparatus for administration to a patient.
